(19) United States
(12) Patent Application Publication
(10) Pub. No.: US 2002/0068014 A1
(43) Pub. Date: Jun. 6, 2002

(54) Antibacterial agents and compositions, methods and systems employing same

(76) Inventors: John Christian Haught, West Chester, OH (US); Gregory Scot Miracle, Hamilton, OH (US); Andre Christian Convents, Cincinnati, OH (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/784,500

(22) Filed: Feb. 15, 2001

Related U.S. Application Data
(60) Provisional application No. 60/183,403, filed on Feb. 18, 2000.

(51) Int. Cl. A01N 1/00; A61L 2/00; A61L 9/00; C11D 3/50; C11D 9/44; C11D 1/00

(52) U.S. Cl. 422/28; 510/101; 510/199

(57) ABSTRACT

The present invention relates to antibacterial agents, more particularly salicylanilide substituted compositions, preferably monosubstituted salicylanilide compositions, most preferably monohalogenated salicylanilide compositions, useful in antibacterial compositions, bacteria-reducing systems, antibacterial products and bacteria-reducing methods.

### CROSS REFERENCE

This application claims the benefit of U.S. Provisional Application No. 60/183,403, filed Feb. 18, 2000.

### FIELD OF THE INVENTION

The present invention relates to antibacterial agents, more particularly salicylanilide substituted compositions, preferably monosubstituted salicylanilide compositions, most preferably monohalogenated salicylanilide compositions, useful in antibacterial compositions, bacteria-reducing systems, antibacterial products and bacteria-reducing methods.

### BACKGROUND OF THE INVENTION

Consumers are very conscientious about cleanliness and/or sanitization, especially when it comes to dishes, utensils, tableware, cookware, and cleaning articles, such as sponges, wash cloths, etc.. that are typically found and/or used in kitchens and bathrooms at home and away from home, such as in restaurants. Other areas of interest are textiles, fabrics and garments that come into contact with consumers. Accordingly, there is a need for a bacteria-reducing system and method that sanitizes such articles, such as garments, textiles, sponges, dishes, tableware, and wash cloths.

The prior art is replete with detergent compositions containing multi-halogenated salicylanilides, especially tribromosalicylanilide and tetrachlorosalicylanilide; see U.S. Pat. Nos. 2,906,711; 3,968,210; 3,989,827; and 4,061,603; German #2,157,209; and British #848,306.

The art teaches that salicylanilides, when used individually, are not effective against gram negative bacteria, only gram positive bacteria (Natarajan et al., 1992, Indian Drugs 29:545-552). Additionally, the art teaches that, in a detergent matrix, mono-halogenated salicylanilides are ineffective; multi-halogenated salicylanilides are required for efficacy. In fact, the art teaches that mono-halogenated salicylanilides are not efficacious in a detergent matrix (Lemair et al., 1961, J. Pharmaceutical Sciences, 50:831-837) with the exception of monohalogenation on the aniline ring of thiosalicylanilides (British patent #1,088,498). The mono halogenated thiosalicylanilides in British #1,088,498 were taught to be effective only against gram-positve bacteria, not against gram-negative bacteria.

Although multi-halogenated salicylanilides are effective in a detergent matrix, these compounds have not enjoyed widespread use due to a variety of reasons, including but not limited to problems encountered in formulating these agents. Accordingly there remains a need for an effective means of formulating salicylanilide type compounds, as well as a need for derivatives that are effective against both gram positive and gram negative bacteria.

### SUMMARY OF THE INVENTION

The present invention meets and fulfills the needs identified above by providing antibacterial compositions, methods and systems that employ certain antibacterial agents, preferably substituted salicylanilide compounds.

Surprisingly, it has been found that certain classes of salicylanilide compounds, which were originally identified as having little or no antibacterial properties, exhibit antibacterial properties in certain formulations.

In one aspect of the present invention, an antibacterial composition comprising an antibacterial agent, preferably a substituted salicylanilide compound of formula I,

[chemical structure]

wherein m is an integer from 0 to 4; n is an integer from 0 to 5; the sum of m+n is greater than zero; a is 0 or 1; b is 0 or 1; g is 0 or 1; when b is 0, one of a and g must be 0; Z and Z′ are independently selected from 0 and S; X and X′, when present, are selected from O, S, and NR¹, where R¹ is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T, when present, is selected from C═O, C═S, S═O, and SO₂; when T is S═O or SO₂, X and X′ may not be S; when either a, b or g is 1 for a radical R—(X)ₐ—(T)_{b}—(X′)_{g}—. R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and neither Z nor Z′ is S, R for that radical may be further selected from the group consisting of F, Cl, Br, I, CN, R₂N→O, NO₂; when Z or Z′ is S, R for that radical may be further selected from the group consisting of CN, R₂N→O, NO₂; when all a, b and g are 0, at least one R must be non-H; further provided that the total number of halogen atoms in the molecule excluding any present in G does not exceed two; G is H, a suitable charge balancing counterion (Mⁿ⁺)_{1/n}, or a cleaveable group selected from the group consisting of Si((O)ₚR²)₃, where p is independently 0 or 1; C(O)_{q}((O)ₚR²)ᵣ, wherein p is independently 0 or 1 and when q is 1, r is 1, and when q is 0, r is 3; R² is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; and

B) at least one additional component selected from the group consisting of:

i) at least 1 wt % of a surfactant, wherein the ratio of the weight of the surfactant divided by the weight of said compound I is greater than or equal to 1.0;

ii) from 0.5% to 90% of a solvent whose Hildebrand solubility parameter d_{S} (cal/cm³)^{1/2} meets the following criterion: 5<d_{S}<20, wherein a 10 wt % aqueous solution of this composition has a pH≧(pKa−1) where pKa is the calculated pKa of the phenol or thiophenol of formula I, or when G is not H, the pKa of the phenol or thiophenol of formula I that results from replacing G with H;

iii) a perfume wherein the perfume has a C log P greater than or equal to 2.0.

iv) an enzyme from 0.001 to 1.0% by weight of the composition;

v) mixtures thereof;

is provided.

In another aspect of the present invention, an antibacterial composition comprising a substituted phenol or thiophenol compound of formula II:

[chemical structure]

wherein m is an integer from 0 to 4; a is 0 or 1; b is 0 or 1; g is 0 or 1; when b is 0, one of a and g must be 0; Z is selected from O and S; X and X′, when present, are selected from O, S, and NR¹; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and Z is O, R for that radical may be further selected from the group consisting of F, Cl, Br, I, CN, R₂N→), NO₂; T, when present, is selected from C═O, C═S, S═O, and SO₂; when T is S═O or SO₂, X and X′ may not be S; Y is a radical comprising at least 1 but no more than 20 carbon atoms and containing a substituent —X″—H, where X″ is selected from O, S, and N—(T′)_{b′}—(X′″)_{a′}—R², where a′ is 0 or 1, b′ is 0 or 1, and X′″, when present, is selected from O, S, and NR²; R² is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T′, when present, is selected from C═O, C═S, and SO₂; when T′ is SO₂, X′″ may not be S; G is H, a suitable charge balancing counterion (Mⁿ⁺)_{1/n}, or a cleaveable group selected from the group consisting of Si((O)ₚR³)₃, where p is independently 0 or 1; C(O)_{q}((O)ₚR³)ᵣ, wherein p is independently 0 or I and when q is 1, r is 1, and when q is 0, r is 3; R³ is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; the parameter d_{Z-H}, the center to center distance from the phenolic oxygen atom or the thiophenolic sulfur atom to the H atom of —X″—H, must satisfy the following criterion in at least ore rotational conformation of the compound II:

1.0 Å≦d_{Z-H}≦4.0 Å;

wherein when G is H or replaced by H, the pKₐ of the substituted phenol or thiophenol, or resulting substituted phenol or thiophenol is from about 5 to about 11; and

B) a surfactant wherein the ratio of the weight of the surfactant divided by the weight of the substituted compound II is greater than or equal to 1.0 and further provided that the surfactant is 1 wt % or greater of the composition; and

C) from 0.5% to 90% of a solvent whose Hildebrand solubility parameter d_{S} (cal/cm³) meets the following criterion: 5<d_{S}<20; and

D) optionally, a surfactant containing a nitrogen head group; quaternary ammonium compound; an amphoteric surfactant; a zwitterionic surfactant; or a primary, secondary, or tertiary amine based surfactant,

further provided that a 10 wt % aqueous solution of this composition has a pH≧(pKₐ−1) where pKₐ is the calculated pKₐ of the substituted phenol or thiophenol or, when G is not

H, the resulting substituted phenol or thiophenol of formula II, is provided.

In yet another aspect of the present invention, a bacteria-reducing system comprising a substituted salicylanilide compound of formula I.

[chemical structure]

wherein m is an integer from 0 to 4; n is an integer from 0 to 5; the sum of m+n is greater than zero; a is 0 or 1; b is 0 or 1; g is 0 or 1; when b is 0, one of a and g must be 0; Z and Z′ are independently selected from O and S; X and X′. when present, are selected from O, S, and NR¹, where R¹ is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T, when present, is selected from C═O, C═S, S═O, and SO₂; when T is S═O or SO₂, X and X′ may not be S; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and neither Z nor Z′ is S, R for that radical may be further selected from the group consisting of F, Cl, Br, I, CN, R₂N→O, NO₂; when Z or Z′ is S, R for that radical may be further selected from the group consisting of CN, R2N→O, NO2; when all a, b and g are 0, at least one R must be non-H; further provided that the total number of halogen atoms in the molecule excluding any present in G does not exceed two; G is H, a suitable charge balancing counterion (Mⁿ⁺)1/n, or a cleaveable group selected from the group consisting of Si((O)ₚR²)₃, where p is independently 0 or 1; C(O)_{q}((O)ₚR²)r, wherein p is independently 0 or 1 and when q is 1, r is 1, and when q is 0, r is 3; R² is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; wherein the bacteria-reducing system reduces bacteria on a substrate where the active is incorporated or added via a solution or lotion, is provided.

In still yet another aspect of the present invention, a method for bacteria-reducing a bacteria-containing substrate comprising contacting the substrate with a bacteria-reducing system according to the present invention, is provided.

In even yet another aspect of the present invention, a bacteria-reduced substrate/article made by the method of present invention, is provided.

In still yet another aspect of the present invention, a bacteria-reducing product comprising an antibacterial composition and/or bacteria-reducing system of the present invention, is provided.

Accordingly, the present invention provides antibacterial compositions, bacteria-reducing systems, bacteria-reducing methods, bacteria-reducing products and bacteria-reduced substrates/articles made by the methods that employ an antibacterial agent, preferably a substituted salicylanilide.

These and other objects, features and advantages will be clear from the following detailed description, examples and appended claims.

All percentages, ratios and proportions herein are on a weight basis based on a neat product unless otherwise indicated. All documents cited herein are hereby incorporated by reference.

### DETAILED DESCRIPTION OF THE INVENTION

Definitions

“System”—“System” as used herein means a complex unity formed of many often, but not always, diverse parts (i.e.. materials, compositions, devices, appliances, procedures, methods, conditions, etc.) subject to a common plan or serving a common purpose.

“Bacteria Reduced Substrate/Article”—“Bacteria Reduced Substrate/Article” as used herein means a substrate/article in which the bacteria present on and/or in the substrate/article have been reduced.

“Substituted”—“Substituted” as used herein means that the organic composition or radical to which the term is applied is:

(a) made unsaturated by the elimination of elements or radical; or

(b) at least one hydrogen in the compound or radical is replaced with a moiety containing one or more (i) carbon, (ii) oxygen, (iii) sulfur, (iv) nitrogen or (v) halogen atoms; or

(c) both (a) and (b).

(i) Moieties which may replace hydrogen as described in (b) immediately above, which contain only carbon and hydrogen atoms are all hydrocarbon moieties including, but not limited to, alkyl, alkenyl, alkynyl, alkyldienyl, cycloalkyl, phenyl, alkyl phenyl, naphthyl, anthryl, phenanthryl, fluoryl, steroid groups, and combinations of these groups with each other and with polyvalent hydrocarbon groups such as alkylen, alkylidene and alkylidyne groups. Specific nonlimiting examples of such groups are:

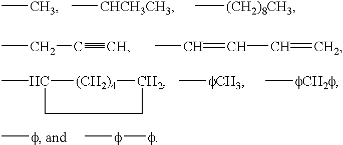

(ii) Moieties containing oxygen atoms which may replace hydrogen as described in (b) immediately above include hydroxy, acyl or keto, ether, epoxy, carboxy, and ester containing groups. Specific nonlimiting examples of such oxygen containing groups are:

—CH₂OH, —CCH₃CH₃OH, —CH₂COOH, —C(O)—(CH₂)₈CH₃, —OCH₂CH₃, ═O, —OH, —CH₂—O—CH₂CH₃, —CH₂—O—(CH₂)₂—OH, —CH₂CH₂COOH, -φOH, -φOCH₂CH₃, -φCH₂OH,

[chemical structure]

(iii) Moieties containing sulfur atoms which may replace hydrogen as described in (b) immediately above include the sulfur-containing acids and acid ester groups, thioether groups, mercapto groups and thioketo groups. Specific nonlimiting examples of such sulfur containing groups are:

—SCH₂CH₃, —CH₂S(CH₂)₄CH₃, —SO₃CH₂CH₃, SO₂CH₂CH₃, —CH₂COSH, —SH, —CH₂SCO, —CH₂C(S)CH₂CH₃, —SO₃H, —O(CH₂)₂C(S)CH₃, ═S, and

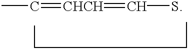

(iv) Moieties containing nitrogen atoms which may replace hydrogen as described in (b) immediately above include amino groups, the nitro group, azo groups, ammonium groups, amide groups, azido groups, isocyanate groups, cyano groups and nitrile groups. Specific nonlimiting examples of such nitrogen containing groups are:

—NHCH₃, —NH₂, —NH₃⁺, —CH₂CONH₂, —CH₂CON₃, —CH₂CH₂CH═NOH, —CN, —CH(CH₃)CH₂NCO, —CH₂NCO, —Nφ, -φN═NφOH, and ≡N.

(v) Moieties containing halogen atoms which may replace hydrogen as described in (b) immediately above include chloro, bromo, fluoro, iodo groups and any of the moieties previously described where a hydrogen or a pendant alkyl group is substituted by a halo group to form a stable substituted moiety. Specific nonlimiting examples of such halogen containing groups are:

—(CH₂)₃COCl, -φF₅, -φCl, —CF₃, and —CH₂φBr.

It is understood that any of the above moieties (i) through (v) can be substituted into each other in either a monovalent substitution or by loss of hydrogen in a polyvalent substitution to form another monovalent moiety which can replace hydrogen in the organic compound or radical.

“φ”—“φ” as used herein represents a phenyl ring.

Antibacterial Agent

The antibacterial agent useful in the present invention preferably comprises a substituted phenol compound of formula II:

[chemical structure]

wherein m is an integer from 0 to 4; a is 0 or 1; b is 0 or 1; g is 0 or 1; when b is 0, one of a and g must be 0; Z is selected from O and S; X and X′, when present, are selected from O, S, and NR¹; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical, R for that radical may be further selected from the group consisting of F, Cl, Br, I, CN, R₂N→O, NO₂; T, when present, is selected from C═O, C═S, S═O, and SO₂; when T is S═O or SO₂, X and X′ may not be S; Y is a radical comprising at least 1 but no more than 20 carbon atoms and containing a substituent —X″—H, where X″ is selected from O, S, and N—(T′)_{b′—(X′″)}_{a′}— R², where a′ is 0 or 1, b′ is 0 or 1, and X′″, when present, is selected from O, S, and NR²; R² is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T′, when present, is selected from C═O, C═S, and SO₂; when T′ is SO₂, X′″ may not be S; G is H, a suitable charge balancing counterion (Mⁿ⁺)1/n, or a cleaveable group selected from the group consisting of Si((O)ₚR³)₃, where p is independently 0 or 1; C(O)_{q}((O)ₚR³)ᵣ, wherein p is independently 0 or 1 and when q is 1, r is 1, and when q is 0, r is 3; R³ is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; the parameter d_{Z-H}, the center to center distance from the phenolic oxygen atom or the thiophenolic sulfur atom to the H atom of —X″—H, must satisfy the following criterion in at least one rotational conformation of the compound II:

1.0 Å≦d_{Z-H}≦4.0 Å;

wherein when G is H or replaced by H, the pKₐ of the substituted phenol or thiophenol, or resulting substituted phenol or thiophenol is from about 5 to about 11.

More preferably, the antibacterial agent comprises the substituted phenol compound of formula II wherein G is C(O)_{q}((O)ₚR¹)ᵣ, wherein p is independently 0 or 1 and when q is 1, r is 1, and when q is 0, r is 3; R¹ is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof.

Even more preferably, the antibacterial agent comprises a salicylanilide compound, preferably a substituted salicylanilide compound having the formula I:

[chemical structure]

wherein m is an integer from 0 to 4; n is an integer from 0 to 5; the sum of m+n is greater than zero; a is 0 or 1; b is 0 or 1; g is 0 or 1; when b is 0, one of a and g must be 0; Z and Z′ are independently selected from O and S; X and X′, when present, are selected from O, S, and NR¹, where R¹ is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T, when present, is selected from C═O, C═S, S═O, and SO₂; when T is S═O or SO₂, X and X′ may not be S; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and neither Z nor Z′ is S, R for that radical may be further selected from the group consisting of F, Cl, Br, I, CN, R₂N→O, NO₂; when Z or Z′ is S, R for that radical may be further selected from the group consisting of CN, R2N→O, NO2; when all a, b and g are 0, at least one R must be non-H; further provided that the total number of halogen atoms in the molecule excluding any present in G does not exceed two; G is H, a suitable charge balancing counterion (Mⁿ⁺)_{1/n}, or a cleaveable group selected from the group consisting of Si((O)ₚR²)₃, where p is independently 0 or 1; C(O)_{q}((O)ₚR²)ᵣ, wherein p is independently 0 or 1 and when q is 1, r is 1, and when q is 0, r is 3; R² is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof.

Specific nonlimiting examples of substitutions that can be made on the salicylanilide rings include the following:

[chemical structure]

In a more preferred embodiment of the present invention, the antibacterial agent comprises a salicylanilide compound having the formula I wherein m is an integer from 0 to 2; n is an integer from 0 to 2; g is 0; Z and Z′ are 0; and T, when present, is selected from C═O and SO₂.

In preferred embodiments of the present invention, the antibacterial agents are selected from monosubstituted salicylanilides, including but not limited to, 5-chlorosalicylanilide, 4-chlorosalicylanilide, 5-iodosalicyanilide, 4-iodosalicylanilide, 5-fluorosalicylanilide, 4-fluorosalicylanilide, 5-cyanosalicylanilide, 4-cyanosalicylanilide, 5-acetylsalicylanilide, and 4-acetylsalicylanilide. The salts of the aforementioned compounds are also a preferred species.

In more preferred embodiments of the present invention, the antibacterial agents are selected from monohalogenated salicylanilide compounds, preferably 4-halosalicylanilide and 5-halosalicylanilide, more preferably 4-chlorosalicylanilide and 5-chlorosalicylanilide, most preferably 5-chlorosalicylanilide.

In highly preferred embodiments of the present invention, the antibacterial agents are selected from monohalogenated salicylanilide compounds, preferably 4-halosalicylanilide and 5-halosalicylanilide, more preferably 4-chlorosalicylanilide and 5-chlorosalicylanilide, most preferably 5-chlorosalicylanilide.

Bacteria-Reducing System

The antibacterial agent of the present invention is useful in reducing bacteria on a substrate/article, when the antibacterial agent is incorporated into a bacteria-reducing system.

Preferably, such a bacteria-reducing system further comprises a surfactant and/or a solvent and/or a perfume and/or an enzyme.

In a preferred embodiment of the present invention, a bacteria-reducing system comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention; and

b) at least 1% by weight of the composition of a surfactant;

wherein the weight ratio of the surfactant to the antibacterial agent is greater than or equal to 1.0, is provided.

Optionally, but preferably, this bacteria-reducing system further comprises a perfume having a C log P greater than or equal to 2.0.

In another preferred embodiment of the present invention, a bacteria-reducing system comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention; and

b) from about 0.5% to about 90% by weight of the composition of a solvent whose Hildebrand solubility parameter δ_{S} (cal/cm³)^{1/2} meets the following criterion: 5<δ_{S}<20;

wherein a 10 wt % aqueous solution of the composition has a pH≧(pKa−1) where pKa is the calculated pKa of the antibacterial agent where —Z—G is —Z—H, is provided.

Optionally, but preferably, this bacteria-reducing system further comprises a perfume having a C log P greater than or equal to 2.0.

In yet another preferred embodiment of the present invention, a bacteria-reducing system comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention; and

b) from about 0.001%, preferably from about 0.01%, more preferably from about 0.1%, most preferably from about 0.5% to about 30%, preferably to about 20%, more preferably to about 10%, most preferably to about 5% by weight of the composition of a perfume wherein the perfume has a C log P greater than or equal to 1.0, preferably 1.5 more preferably 2.0, is provided.

In still yet another embodiment of the present invention, a bacteria-reducing system comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention;

b) at least 1% by weight of the composition of a surfactant;

wherein the weight ratio of the surfactant to the antibacterial agent is greater than or equal to 1.0; and

c) from about 0.5% to about 90% by weight of the composition of a solvent whose Hildebrand solubility parameter δ_{S} (cal/cm³)^{1/2} meets the following criterion: 5<δ_{S}<20;

wherein a 10wt % aqueous solution of the composition has a pH≧(pKa−1) where pKa is the calculated pKa of the antibacterial agent where -Z-G is -Z-H, is provided.

The substrate may be a hard or soft substrate. Hard substrates are selected from the group consisting of: utensils, dishes, countertops, cookware, pots, pans, skillets, baby bottles, baby nipples, glassware, dentures, kitchen cutting boards made of wood or any other suitable material, and mixtures thereof. Soft substrates are selected from the group consisting of: textiles, fabrics, garments, sponges, wash cloths, brushes, gloves, scouring pads, reusable wipes, animal and human skin (i.e., personal cleansing applications) and mixtures thereof. In addition to these substrates, the substrates may include food articles, such as fruits, meats and liquids, such as water.

The bacteria on and/or in the substrate is preferably selected from the group consisting of: Staphylococcus aureus, Staphylococcus haemolyticus, Staphylococcus capitis, Klebsiella pneumoniae, Proteus mirabilis, Serratia marcescens, Staphylococcus epidermis, Salmonella typhimurium, Shigella dysenteriae, Streptococcus faecalis, Streptococcus pyogenes, Cornybacterium xerosis, Micrococcus varians, Micrococcus luteus, Peptostreptococcus anaerobius, Propionibacterium acnes, Propionibacterium avidum, Propionibacterium granulosum, Escherichia coli, Salmonella choleraesius, Listeria monocytogenes, Enterococcus hirae and mixtures thereof more preferably. Escherichia coli, Salmonella choleraesius, Listeria monocytogenes and mixtures thereof, and most preferably Escherichia coli, Salmonella choleraesius and mixtures thereof.

Antibacterial Composition

The antibacterial agent of the present invention is preferably incorporated with one or more additional adjunct ingredients into one or more antibacterial compositions. Preferably, the antibacterial compositions of the present invention are free of aminopolyureylene resin because unlike prior art antibacterial compositions the antibacterial agents and systems and compositions of the present invention exhibit relatively dilute efficacy and do not require a resin to attach the antibacterial agents to a substrate to be efficacious.

These one or more additional adjunct ingredients are determined according to the type of composition that the antibacterial agent is to be incorporated into and/or the type of use of the antibacterial composition.

The antibacterial agent can be incorporated into a range of different compositions and/or products including, but not limited to, liquid dishwashing detergent compositions, heavy duty detergent compositions, automatic dishwashing compositions, hard surface cleaning compositions, home care compositions, fabric care compositions and dryer-added compositions. These compositions and/or products may be in any form known to those skilled in the art. For example, the compositions and/or products may be in liquid, granular, powder, tablet, paste, foam and bars. These compositions and/or products may be neat or releasably absorbed or adsorbed on to a substrate, such as a woven or non-woven filament substrate.

For example, an antibacterial composition in accordance with the present invention preferably comprises an antibacterial agent of the present invention with a surfactant, preferably at least 1% by weight of the composition of a surfactant, and/or a solvent, preferably from about 0.5% to 90% by weight of the composition of a solvent whose Hildebrand solubility parameter δ_{S} (cal/cm³)^{1/2} meets the following criterion: 5<δ_{S}<20, and wherein a 10wt % aqueous solution of the composition has a pH≧(pKa−1) where pKa is the calculated pKa of the antibacterial agent where —Z—G is —Z—H, and/or a perfume, preferably a perfume wherein the perfume has a C log P greater than or equal to 2.0, and/or an enzyme. Optionally, but preferably, the composition comprises one or more additional detergent adjunct ingredients selected from the group consisting of: bleaching systems, brighteners, builders, chelants, soil release polymers, dye transfer inhibiting agents.

In a preferred embodiment of the present invention, an antibacterial composition comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention; and

b) at least 1% by weight of the composition of a surfactant;

wherein the weight ratio of the surfactant to the antibacterial agent is greater than or equal to 1.0, is provided.

Optionally, but preferably, this antibacterial composition further comprises a perfume oil, preferably a hydrophobic perfume oil.

In another preferred embodiment of the present invention, an antibacterial composition comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention; and

b) from about 0.5% to about 90% by weight of the composition of a solvent whose Hildebrand solubility parameter δ_{S} (cal/cm³)^{1/2} meets the following criterion: 5<δ_{S}<20;

wherein a 10 wt % aqueous solution of the composition has a pH≧(pKa−1) where pKa is the calculated pKa of the antibacterial agent where —Z—G is —Z—H, is provided

Optionally, but preferably, this antibacterial composition further comprises a perfume oil, preferably a hydrophobic perfume oil.

In yet another preferred embodiment of the present invention, an antibacterial composition comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention; and

b) from about 0.001%, preferably from about 0.01%, more preferably from about 0.1%, most preferably from about 0.5% to about 30%, preferably to about 20%, more preferably to about 10%, most preferably to about 5% by weight of the composition of a perfume wherein the perfume has a C log P greater than or equal to 1.0, preferably 1.5 more preferably 2.0, is provided.

In still yet another embodiment of the present invention, an antibacterial composition comprising:

a) an effective amount, preferably from about 0.001%, more preferably from about 0.01%, even more preferably from about 0.05% to about 15%, more preferably to about 10%, even more preferably to about 5%, most preferably to about 2.5% by weight of the system of an antibacterial agent of the present invention;

b) at least 1% by weight of the composition of a surfactant;

wherein the weight ratio of the surfactant to the antibacterial agent is greater than or equal to 1.0; and

c) from about 0.5% to about 90% by weight of the composition of a solvent whose Hildebrand solubility parameter δ_{S} (cal/cm³)^{1/2} meets the following criterion: 5<δ_{S}<20;

wherein a 10 wt % aqueous solution of the composition has a pH≧(pKa−1) where pKa is the calculated pKa of the antibacterial agent where —Z—G is —Z—H, is provided.

Dilute Efficacy Test Protocol

The antibacterial compositions and/or antibacterial products and/or bacteria-reducing systems, preferably antibacterial compositions of the present invention exhibit relatively dilute efficacy in reducing bacteria from substrates/articles as measured by this Dilute Efficacy Test.

To determine whether an antibacterial composition and/or antibacterial product and/or bacteria-reducing system comprising an antibacterial agent of the present invention satisfies this Dilute Efficacy Test, a Dilute Efficacy Test Protocol has been established. This Dilute Efficacy Test Protocol is a modification of AOAC Official Method 960.09.

Step 1: Dilute a 0.25% by weight antibacterial agent containing composition or product or system with hard water (14 grains per gallon) to a 1:3 dilution in the presence of 5% soil (horse serum commercially available from Sigma).

Step 2: Contact at 25° C. for 30 minutes the dilute solution from Step I with 10⁶ cfu/ml of a one of the three gram negative bacteria: Salmonella choleraesuis ATCC# 10708, Klebsiella pneumoniae ATCC# 4352 and Escherichia coli ATCC# 11229, the nutrient medium that the bacteria are grown in is Nutrient agar or Trypticase soy broth, and the incubation conditions for the bacteria are 37° C. for 24 hours.

Step 3: Neutralize the antibacterial agent by adding a 1:10 dilution of neutralizer broth (1×concentration of DIE Neutralizing Broth commercially available from Difco Laboratories, 9.5% Tween 80 and 2.5% sodium thiosulfate) to the solution of Step 2.

Step 4: Determine the log reduction by the plate count method or using a bactometer to determine the log remaining bacteria.

Step 5: If the log reduction for Salmonella choleraesuis ATCC# 10708 or Klebsiella pneumoniae ATCC# 4352 or Escherichia coli ATCC# 11229 is 2 or greater, preferably 3 or greater, more preferably 4 or greater, most preferably 5 or greater, the antibacterial agent containing composition, product or system exhibits Dilute Efficacy within the scope of the present invention.

Adjunct Ingredients

In addition to the antibacterial agent, one or more adjunct ingredients as described below may optionally, but preferably, be included in the compositions, products and/or systems comprising the antibacterial agent.

Examples of suitable adjunct ingredients include, but are not limited to, builders, bleaches, bleach activators, bleach catalysts, enzyme stabilizing systems, chelants, optical brighteners, soil release polymers, dye transfer agents, dispersants, suds suppressors, dyes, colorants, filler salts, hydrotropes, photoactivators, fluorescers, fabric conditioners, hydrolyzable surfactants, perservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments and pH control agents as described in U.S. Pat. Nos. 5,705,464, 5,710,115, 5,698,504, 5,695,679, 5,686,014 and 5,646,101. Specific cleaning adjunct materials are exemplified in detail hereinafter.

Preferred Adjunct Ingredients

Surfactants—A wide range of surfactants can be used in the compositions of the present invention.

Surfactants included in the fully-formulated compositions afforded by the present invention comprise at least 0.01%, preferably at least about 0.1%, more preferably at least about 0.5%, even more preferably at least about 1%, most preferably at least about 3% to about 80%, more preferably to about 60%, most preferably to about 50% by weight of composition depending upon the particular surfactants used and the desired effects to be achieved.

The surfactant can be nonionic, anionic, amphoteric, amphophilic, zwitterionic, cationic, semi-polar nonionic, and mixtures thereof, nonlimiting examples of which are disclosed in U.S. Pat. Nos. 5,707,950 and 5,576,282. A typical listing of anionic, nonionic, amphoteric and zwitterionic classes, and species of these surfactants, is given in U.S. Pat. No. 3,664,961 issued to Norris on May 23, 1972. Preferred compositions comprise nonionic surfactants and/or mixtures of nonionic surfactants with other surfactants, especially anionic surfactants.

i. Nonionic Surfactant

Suitable nonionic surfactants are generally disclosed in U.S. Pat. No. 3,929,678, Laughlin et al., issued Dec. 30, 1975, and U.S. Pat. No. 4,285,841, Barrat et al, issued Aug. 25, 1981. Exemplary, non-limiting classes of useful nonionic surfactants include: C₈-C₁₈ alkyl ethoxylates (“AE”), with EO about 1-22, including the so-called narrow peaked alkyl ethoxylates and C₆-C₁₂ alkyl phenol alkoxylates (especially ethoxylates and mixed ethoxy/propoxy), alkyl dialkyl amine oxide, alkanoyl glucose amide, and mixtures thereof.

If nonionic surfactants are used, the compositions of the present invention will preferably contain from about 1% to about 80%, more preferably from about 1% to about 60%, most preferably from about 1% to about 50% by weight of nonionic surfactant.

Preferred nonionic surfactants include, but are not limited to, the ethoxylated alcohols and ethoxylated alkyl phenols of the formula R(OC₂H₄)ₙOH, wherein R is selected from the group consisting of aliphatic hydrocarbon radicals containing from about 8 to about 15 carbon atoms and alkyl phenyl radicals in which the alkyl groups contain from about 8 to about 12 carbon atoms, and the average value of n is from about 5 to about 15. These surfactants are more fully described in U.S. Pat. No. 4,284,532. Leikhim et al, issued Aug. 18, 1981. Particularly preferred are ethoxylated alcohols having an average of from about 9 to abut 15 carbon atoms in the alcohol and an average degree of ethoxylation of from about 5 to about 15 moles of ethylene oxide per mole of alcohol.

Other nonionic surfactants for use herein include:

The polyethylene, polypropylene, and polybutylene oxide condensates of alkyl phenols. Commercially available nonionic surfactants of this type include Igepal® CO-630, marketed by the GAF Corporation; and Triton® X45, X-114, X-100, and X-102, all marketed by the Rohm & Haas Company. These compounds are commonly referred to as alkyl phenol alkoxylates, (e.g., alkyl phenol ethoxylates).

The condensation products of aliphatic alcohols with from about 1 to about 25 moles of ethylene oxide. Examples of commercially available nonionic surfactants of this type include Tergitol® 15-S-9 (the condensation product of C₁₁-C₁₅ linear secondary alcohol with 9 moles ethylene oxide), Tergitol® 24-L-6 NMW (the condensation product of C₁₂-C₁₄ primary alcohol with 6 moles ethylene oxide with a narrow molecular weight distribution), both marketed by Union Carbide Corporation; Neodol® 45-9 (the condensation product of C₁₄-C₁₅ linear alcohol with 9 moles of ethylene oxide), Neodol® 23-9 (the condensation product of C₁₂-C₁₃ linear alcohol with 9 moles of ethylene oxide); Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 45-4 (the condensation product of C₁₄-C₁₅ linear alcohol with 4 moles of ethylene oxide), marketed by Shell Chemical Company, and Kyro® EOB (the condensation product of C₁₃-C₁₅ alcohol with 9 moles ethylene oxide), marketed by The Procter & Gamble Company. Other commercially available nonionic surfactants include Dobanol 91-8® marketed by Shell Chemical Co. and Genapol UD-080® marketed by Hoechst. This category of nonionic surfactant is referred to generally as “alkyl ethoxylates.”

The condensation products of ethylene oxide with a hydrophobic base formed by the condensation of propylene oxide with propylene glycol. Examples of compounds of this type include certain of the commercially-available Pluronic® surfactants, marketed by BASF.

The condensation products of ethylene oxide with the product resulting from the reaction of propylene oxide and ethylenediamine. Examples of this type of nonionic surfactant include certain of the commercially available Tetronic® compounds, marketed by BASF.

Semi-polar nonionic surfactants, especially water-soluble amine oxides. Preferably, these amine oxide surfactants include C₁₀-C₁₈ alkyl dimethyl amine oxides and C₈-C₁₂ alkoxy ethyl dihydroxy ethyl amine oxides.

Alkylpolysaccharides disclosed in U.S. Pat. No. 4,565,647, Llenado, issued Jan. 21, 1986, having a hydrophobic group containing from about 6 to about 30 carbon atoms, and alkylpolyglycosides disclosed in EP-B 070 077 EP-B-075 996 and EP-B 094 118.

Fatty acid amide surfactants having the formula:

[chemical structure]

wherein R⁶ is an alkyl group containing from about 7 to about 21 (preferably from about 9 to about 17) carbon atoms and each R⁷ is selected from the group consisting of hydrogen, C₁-C₄ alkyl, C₁-C₄ hydroxyalkyl, and —(C²H₄O)ₓH where x varies from about 1 to about 3. Preferred amides are C₈-C₂₀ ammonia amides, monoethanolamides, dietha-nolamides, and isopropanolamides.

These and other nonionic surfactants are well known in the art, being described in more detail in Kirk Othmer's Encyclopedia of Chemical Technology, 3rd Ed., Vol. 22, pp. 360-379, “Surfactants and Detersive Systems”, incorporated by reference herein.

ii. Anionic Surfactant

Generally speaking, anionic surfactants useful herein are disclosed in U.S. Pat. No. 4,285,841, Barrat et al. issued Aug. 25, 1981, and in U.S. Pat. No. 3,919,678, Laughlin et al, issued Dec. 30, 1975, both incorporated herein by reference.

Anionic surfactants include, but are not limited to, linear alkylbenzene sulfonate, alpha olefin sulfonate, paraffin sulfonates, alkyl ester sulfonates, alkyl sulfates, alkyl alkoxy sulfate, alkyl sulfonates, alkyl alkoxy carboxylate, alkyl alkoxylated sulfates, sarcosinates, taurinates, and mixtures thereof. More preferably, the anionic surfactants include, but are not limited to, C₁l-C₁₈ alkyl benzene sulfonates (LAS) and primary, branched-chain and random C₁₀-C₂₀ alkyl sulfates (AS), the C₁₀-C₁₈ secondary (2.3) alkyl sulfates of the formula CH₃(CH₂)ₓ(CHOSO₃⁻M⁺) CH₃ and CH₃ (CH₂)_{y}(CHOSO₃⁻M⁺) CH₂CH₃ where x and (y+1) are integers of at least about 7, preferably at least about 9, and M is a water-solubilizing cation, especially sodium, unsaturated sulfates such as oleyl sulfate, the C₁₀-C₁₈ alkyl alkoxy sulfates (“AEₓS”; especially EO 1-7 ethoxy sulfates, such as 1.8 and 1.1), C₁₀-C₁₈ alkyl alkoxy carboxylates (especially the EO 1-11 ethoxycarboxylates), the C₁₀-C₁₈ sulfated glycerol ethers, the C₁₀-C₁₈ sulfated alkyl polyglycosides, and C₁₂-C₁₈ alpha-sulfonated fatty acid esters.

Useful anionic surfactants include the water-soluble salts, particularly the alkali metal, ammonium and alkylolammonium (e.g., monoethanolammonium or triethanolammonium) salts, of organic sulfuric reaction products having in their molecular structure an alkyl group containing from about 10 to about 20 carbon atoms and a sulfonic acid or sulfuric acid ester group. (Included in the term “alkyl” is the alkyl portion of aryl groups.) Examples of this group of synthetic surfactants are the alkyl sulfates, especially those obtained by sulfating the higher alcohols (C₈-C₁₈ carbon atoms) such as those produced by reducing the glycerides of tallow or coconut oil. Especially valuable are linear straight chain alkylbenzene sulfonates in which the average number of carbon atoms in the alkyl group is from about 11 to 13, abbreviated as C₁₁-C₁₃LAS.

Further examples are described in “Surface Active Agents and Detergents” (Vol. I and II by Schwartz, Perry and Berch) and in U.S. Pat. No. 3,929,678, issued Dec. 30, 1975 to Laughlin, et al. at Column 23, line 58 through Column 29, line 23 (herein incorporated by reference).

Highly preferred anionic surfactants include alkyl alkoxylated sulfate surfactants hereof are water soluble salts or acids of the formula RO(A)ₘSO3M wherein R is an unsubstituted C₁₀-C₂₄ alkyl or hydroxyalkyl group having a C₁₀-C₂₄ alkyl component, preferably a C₁₂-C₂₀ alkyl or hydroxyalkyl, more preferably C₁₂-C₁₈ alkyl or hydroxyalkyl, A is an ethoxy or propoxy unit, m is greater than zero, typically between about 0.5 and about 6, more preferably between about 0.5 and about 3, and M is H or a cation which can be, for example, a metal cation (e.g., sodium, potassium, lithium, calcium, magnesium. etc.), ammonium or substituted-ammonium cation. Alkyl ethoxylated sulfates as well as alkyl propoxylated sulfates are contemplated herein. Specific examples of substituted ammonium cations include methyl-, dimethyl, trimethyl-ammonium cations and quaternary ammonium cations such as tetramethyl-ammonium and dimethyl piperdinium cations and those derived from alkylamines such as ethylamine, diethylamine, triethylamine, mixtures thereof, and the like. Exemplary surfactants are C₁₂-C₁₈ alkyl polyethoxylate (1.0) sulfate (C₁₂-C₁₈E(1.0)M), C₁₂-C₁₈ alkyl polyethoxylate (2.25) sulfate (C₁₂-C₁₈E(2.25)M), C₁₂-C₁₈ alkyl polyethoxylate (3.0) sulfate (C₁₂-C₁₈E(3.0)M), and C₁₂-C₁₈ alkyl polyethoxylate (4.0) sulfate (C₁₂-C₁₈E(4.0)M), wherein M is conveniently selected from sodium and potassium.

When included therein, the compositions of the present invention typically comprise from about 0.5%, preferably from about 3%, more preferably from about 5%, most preferably from about 10% to about 90%, preferably to about 50%, more preferably to about 20%, most preferably to about 10% by weight of such anionic surfactants.

iii. Cosurfactants

The compositions of the present invention may further comprise, especially when anionic surfactants are present, a cosurfactant selected from the group of primary or tertiary amines. Suitable primary amines for use herein include amines according to the formula:

R₁NH₂

wherein R₁ is a C₆-C₁₂, preferably C₆-C₁₀ alkyl chain, or R₄X(CH₂)n, wherein X is —O—, —C(O)NH— or —NH—, R₄ is a C₆-C₁₂ alkyl chain n is between 1 to 5, preferably 3. R₁ alkyl chains may be straight or branched and may be interrupted with up to 12, preferably less than 5 ethylene oxide moieties; or

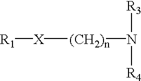

wherein R₁ is a C₆-C₁₂ alkyl group; n is from about 1 to 5, preferably 2 to about 4, more preferably 3. X is a bridging group which is selected from —NH—, —C(O)NH—, —C(O)O—, or —O— or X can be absent; and R₃ and R₄ are individually selected from H, C₁-C₄ alkyl, or (CH₂—CH₂—O(R₅)) wherein R₅ is H or methyl;

Preferred amines according to the formula herein above are n-alkyl amines. Suitable amines for use herein may be selected from 1-hexylamine, 1-octylamine, 1-decylamine and laurylamine. Other preferred primary amines include C8-C10 oxypropylamine, octyloxypropylamine, 2-ethylhexyl-oxypropylamine, lauryl amido propylamine and amido propylamine. The most preferred amines for use in the compositions herein are 1-hexylamine, 1-octylamine, 1-decylamine, 1-dodecylamine. Especially desirable are n-dodecyldimethylamine and bishydroxyethylcoconutalkylamine and oleylamine 7 times ethoxylated, lauryl amido propylamine and cocoamido propylamine.

Preferred amines include the following:

R₁—(CH₂)₂—NH₂ (1)

R₁—O—(CH₂)₃—NH₂ (2)

R₁—C(O)—NH—(CH₂)₃—N(CH₃)₂ (3)

[chemical structure]

wherein R₁ is a C₆-C₁₂ alkyl group and R₅ is H or CH₃.

In a highly preferred embodiment, the amine is described by the formula:

R₁—C(O)—NH—(CH₂)₃—N(CH₃)₂

wherein R₁ is C₈-C₁₂ alkyl.

Particularly preferred amines include those selected from the group consisting of octyl amine, hexyl amine, decyl amine, dodecyl amine, C₈-C₁₂ bis(hydroxyethyl)amine, C₈-C₁₂ bis(hydroxyisopropyl)amine, and C₈-C₁₂ amido-propyl dimethyl amine, and mixtures.

If utilized the detersive amines comprise from about 0.1% to about 10%, preferably from about 0.5% to about 5%, by weight of the composition.

iv. Quaternary Ammonium Surfactants

Suitable quaternary ammonium surfactants include, but are not limited to, quaternary ammonium surfactants having the formula:

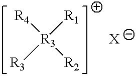

wherein R₁ and R₂ are individually selected from the group consisting of C₁-C₄ alkyl, C₁-C₄ hydroxy alkyl, benzyl, and —(C₂H₄O)ₓH where x has a value from about 2 to about 5; X is an anion; and (1) R₃ and R₄ are each a C₆-C₁₄ alkyl or (2) R₃ is a C₆-C₁₈ alkyl, and R₄ is selected from the group consisting of C₁-C₁₀ alkyl, C₁-C₁₀ hydroxy alkyl, benzyl, and —(C₂H₄O)ₓH where x has a value from 2 to 5.

Preferred quaternary ammonium surfactants are the chloride, bromide, and methylsulfate salts. Examples of preferred mono-long chain alkyl quaternary ammonium surfactants are those wherein R₁, R₂, and R₄ are each methyl and R₃ is a C₈-C₁₆ alkyl; or wherein R₃ is C₈₋₁₈ alkyl and R₁, R₂, and R₄ are selected from methyl and hydroxy-alkyl moieties. Lauryl trimethyl ammonium chloride, myristyl trimethyl ammonium chloride, palmityl trimethyl ammonium chloride, coconut trimethylammonium chloride, coconut trimethylammonium methylsulfate, coconut dimethyl-monohydroxyethyl-ammonium chloride, coconut dimethyl-monohydroxyethylammonium methylsulfate, steryl dimethyl-monohydroxy-ethylammonium chloride, steryl dimethylmonohydroxy-ethylammonium methylsulfate, di- C₁₂-C₁₄ alkyl dimethyl ammonium chloride, and mixtures thereof are particularly preferred. ADOGEN 412™, a lauryl trimethyl ammonium chloride commercially available from Witco, is also preferred. Even more highly preferred are the lauryl trimethyl ammonium chloride and myristyl trimethyl ammonium chloride.

Alkoxylated quaternary ammonium (AQA) surfactants useful in the present invention are of the general formula:

[chemical structure]

wherein R¹ is an alkyl or alkenyl moiety containing from about 8 to about 18 carbon atoms, preferably 10 to about 16 carbon atoms, most preferably from about 10 to about 14 carbon atoms; R² and R^{3′} are each independently alkyl groups containing from one to about three carbon atoms, preferably methyl; R³ and R⁴ can vary independently and are selected from hydrogen (preferred), methyl and ethyl, X⁻ is an anion such as chloride, bromide, methylsulfate, sulfate, or the like, to provide electrical neutrality; A is selected from C₁-C₄ alkoxy, especially ethoxy (i.e., —CH₂CH₂O—), propoxy, butoxy and mixtures thereof; and for formula I, p is from 2 to about 30, preferably 2 to about 15, most preferably 2 to about 8; and for formula II, p is from 1 to about 30, preferably 1 to about 4 and q is from 1 to about 30, preferably 1 to about 4, and most preferably both p and q are 1.

Other quaternary surfactants include the ammonium surfactants such as alkyldimethylammonium halogenides, and those surfactants having the formula:

[R²(OR³)_{y}][R⁴(OR³)_{y}]₂R⁵N⁺X⁻

wherein R² is an alkyl or alkyl benzyl group having from about 8 to about 18 carbon atoms in the alkyl chain, each R³ is selected from the group consisting of —CH₂CH₂—, —CH₂CH(CH₃)—, —CH₂CH(CH₂OH)—, —CH₂CH₂CH₂—, and mixtures thereof; each R⁴ is selected from the group consisting of C₁-C₄ alkyl, C₁-C₄ hydroxyalkyl, benzyl, ring structures formed by joining the two R⁴ groups, —CH₂CHOHCHOHCOR⁶CHOH—CH₂OH wherein R⁶ is any hexose or hexose polymer having a molecular weight less than about 1000, and hydrogen when y is not O; R⁵ is the same as R⁴ or is an alkyl chain wherein the total number of carbon atoms of R² plus R⁵ is not more than about 18; each y is from 0 to about 10 and the sum of the y values is from 0 to about 15: and X is any compatible anion.

v. Fatty Acid

Suitable fatty acids that can be incorporated into the compositions of the present invention in addition to surfactants, include, but are not limited to, saturated and/or unsaturated fatty acids obtained from natural sources or synthetically prepared. Examples of fatty acids include capric, lauric, myristic, palmitic, stearic, arachidic, and behenic acid. Other fatty acids include palmitoleic, oleic, linoleic, linolenic, and ricinoleic acid.

vi. Cationic/Amphoteric Surfactants

Non-quaternary, cationic surfactants can also be included in the compositions of the present invention. Cationic surfactants useful herein are described in U.S. Pat. No. 4,228,044, Cambre, issued Oct. 14, 1980.

Amphoteric surfactants can be incorporated into the compositions hereof. These surfactants can be broadly described as aliphatic derivatives of secondary or tertiary amines, or aliphatic derivatives of heterocyclic secondary and tertiary amines in which the aliphatic radical can be straight chain or branched. U.S. Pat. No. 3,929,678 to Laughlin et al., issued Dec. 30, 1975 at column 19, lines 18-35 discloses examples of amphoteric surfactants.

Further examples of suitable amphoteric surfactants are given in “Surface Active Agents and Detergents” (Vol. I and II by Schwartz, Perry and Berch), hereby incorporated by reference.

Preferably the cationic and/or amphoteric surfactants, when present, are present in the composition in an effective amount, more preferably from about 0.1% to about 20%, even more preferably about 0.1% to about 15%, even more preferably still from about 0.5% to about 10%,by weight.

ix. Biodegradably Branched Surfactants

The compositions of the present invention may also include biodegradably branched and/or crystallinity disrupted and/or mid-chain branched surfactants or surfactant mixtures. These surfactants are more fully disclosed in WO98/23712 A published Jun. 4, 1998; WO97/38957 A published Oct. 23, 1997; WO97/38956 A published Oct. 23, 1997; WO97/39091 A published Oct. 23, 1997; WO97/39089 A published Oct. 23, 1997; WO97/39088 A published Oct. 23, 1997; WO97/39087 A1 published Oct. 23, 1997; WO97/38972 A published Oct. 23, 1997; WO 98/23566 A Shell, published Jun. 4, 1998; technical bulletins of Sasol; and the following pending patent applications assigned to Procter & Gamble: U.S. patent application Ser. Nos. 09/170,711 and 09/170,694.

Perfumes—The term “perfume” as used herein is defined as “a ‘fragrance raw material’ or mixture of ‘fragrance raw materials’ which can be artfully combined to impart a pleasurable scent, odor, essence, or fragrance characteristic”. For the purposes of the present invention “fragrance raw materials” are herein defined as compounds having a molecular weight of at least 100 g/mol and which are useful in imparting an odor, fragrance, essence, or scent either alone or in combination with other “fragrance raw materials”.

Typically “fragrance raw materials” comprise inter alia alcohols, ketones, aldehydes, esters, ethers, nitriles, and cyclic and acyclic alkenes such as terpenes. A listing of common “fragrance raw materials” can be found in various reference sources, for example, “Perfume and Flavor Chemicals”, Vols. I and II; Steffen Arctander Allured Pub. Co. (1994) and “Perfumes: Art, Science and Technology”; Muller, P. M. and Lamparsky, D., Blackie Academic and Professional (1994) both incorporated herein by reference.

Examples of perfume ingredients useful in the perfumes of the subject invention compositions include, but are not limited to, hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma.

Additional fragrance materials of synthetic or natural origin which may be included in the perfume, if desired, include, but are not limited to, orange oil; lemon oil; grapefruit oil; bergamot oil; clove oil; dodecalactone gamma; methyl-2-(2-pentyl-3-oxo-cyclopentyl) acetate; beta-naphthol methylether; methyl-beta-naphthylketone; coumarin; decylaldehyde; benzaldehyde; 4-tert-butylcyclohexylacetate; alpha,alpha-dimethylphenethyl acetate; methylphenylcarbinyl acelate; Schiffs base of 4-(4-hydroxy4-methylpentyl)-3-cyclohexene-1-carboxaldehyde and methyl anthranilate; cyclic ethyleneglycol diester of tridecandioic acid; 3,7-dimethyl-2,6-octadiene-1-nitrile; ionone gamma methyl; ionone alpha; ionone beta; petitgrain; methyl cedrylone; 7-acetyl-1,2,3,4,5,6,7,8-octahydro-1,1,6,7-tetramethyl-naphthalene; ionone methyl; methyl-1,6,10-trimethyl-2,5,9-cyclododecatrien-1-yl ketone; 7-acetyl-1,1,3,4,4,6-hexamethyl tetralin; 4-acetyl-6-tert-butyl-1,1-dimethyl indane; benzophenone; 6-acetyl-1,1,2,3,3,5-hexamethyl indane; 5-acetyl-3-isopropyl-1,1,2,6-tetramethyl indane; 1-dodecanal; 7-hydroxy-3,7-dimethyl octanal; 10-undecen-1-al; iso-hexenyl cyclohexyl carboxaldehyde; formyl tricyclodecan; cyclopentadecanolide; 16-hydroxy-9-hexadecenoic acid lactone; 1,3,4,6,7,8-hexahydro4,6,6,7,8,8-hexamethylcyclopenta-gamma-2-benzopyrane; ambroxane; dodecahydro-3a,6,6,9a-tetramethyinaphtho-[2,1b]furan; cedrol; 5-(2,2,3-trimethylcyclopent-3-enyl)-3-methylpentan-2-ol; 2-ethyl4-(2,2,3-trimethyl-3-cyclopenten-1-yl)-2-buten-1-ol; caryophyllene alcohol; cedryl acetate; para-tert-butylcyclohexyl acetate; patchouli; olibanum resinoid; labdanum; vetivert; copaiba balsam; fir balsam; and condensation products of: hydroxycitronellal and methyl anthranilate; hydroxycitronellal and indol; phenyl acetaldehyde and indol; 4-(4-hydroxy4-methyl pentyl)-3-cyclohexene-1-carboxaldehyde and methyl anthranilate.

More examples of perfume components are geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzyl carbinol; trichloromethylphenylcarbinyl methylphenylcarbinyl acetate; isononyl acetate; vetiveryl acetate; vetiverol; 2-methyl-3-(p-pert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methylether; isolongifolanone; aubepine nitrile; aubepine; heliotropine; eugenol; vanillin; diphenyl oxide; hydroxycitronellal ionones; methyl ionones; isomethyl ionomes; irones, cis-3-hexenol and esters thereof; indane musk fragrances; tetralin musk fragrances; isochroman musk fragrances; macrocyclic ketones; macrolactone musk fragrances; ethylene brassylate.

Definition of the C log P of a perfume: As used herein, the C log P of a perfume, (C log P)ₚ, is calculated as the weighted average of the C log P values of the n individual fragrance raw materials, (C log P)ᵢ, that comprise the perfume, according to the formula:

$${( C log P )}_{p} = {\sum }_{i = 1}^{n} ( \frac{{w}_{i}}{{w}_{p}} ) {( C log P )}_{i}$$

wherein wᵢ is the weight of the nth fragrance raw material and wₚ, the weight of the perfume, is the sum of the weights of the n individual fragrance raw materials according to the formula:

$${w}_{p} = {\sum }_{i = 1}^{n} {w}_{i}$$

All fragrance raw materials present in an amount such that (wᵢ/wp)>0.01 constitute the n fragrance raw materials of the perfume for the purpose of determining (C log P)ₚ.

Solvents—The compositions herein may comprise as an optional, but preferable ingredient, a solvent or mixtures thereof. When used, solvents will, advantageously, give an enhanced performance to the compositions of the present invention. Suitable solvents for incorporation in the compositions according to the present invention include propylene glycol derivatives such as n-butoxypropanol or n-butoxypropoxypropanol, water-soluble CARBITOL® solvents or water-soluble CELLOSOLVE® solvents. Water-soluble CARBITOL® solvents are compounds of the 2-(2-alkoxyethoxy)ethanol class wherein the alkoxy group is derived from ethyl, propyl or butyl. A preferred water-soluble carbitol is 2-(2-butoxyethoxy)ethanol also known as butyl carbitol. Water-soluble CELLOSOLVE® solvents are compounds of the 2-alkoxyethoxyethanol class, with 2-butoxyethoxyethanol being preferred. Preferred solvents for use herein are ethanolamines and alcohols. Most preferrably, the solvents for use in the present compositions are n-butoxypropoxypropanol, butyl carbitol®, monoethanolamine(MEA), diethanolamine, triethanolamine, benzyl alcohol, methanol, ethanol, isopropyl alcohol and diols such as 2-ethyl-1,3-hexanediol and 2,2,4-trimethyl-1,3-pentanediol and mixture thereof. Preferred solvents are typically utilized in the present compositions at a level of from about 0% to about 30%. preferably from about 0.5% to about 25%, more preferably from about 0.5% to about 20% by weight of the composition.

Other useful solvents for use in the present compositions include a poly(alkylene glycol) alkyl ether, as defined herein after, or mixtures thereof.

Typically, where present the composition may comprise a poly(alkylene glycol) alkyl ether or a mixture thereof at a level of from 0.001% to 10%, preferably from 0.005% to 2%, more preferably from 0.01% to 1%, even more preferably from 0.05% to 0.5% and most preferably from 0.08% to 0.4% by weight of the total composition.

Suitable poly(alkylene glycol) alkyl ethers for use herein are according the following formula:

R¹—O—(CH₂—CHR²O)ₙ-R³

wherein R¹ and R² each independently are hydrogen or a substituted or unsubstituted, saturated or unsaturated, linear or branched hydrocarbon chain having from 1 to 30 carbon atoms or a hydroxy bearing linear or branched hydrocarbon chain having from 1 to 30 carbon atoms, R³ is a substituted or unsubstituted, saturated or unsaturated, linear or branched hydrocarbon chain having from 1 to 30 carbon atoms or a hydroxy bearing linear or branched hydrocarbon chain having from 1 to 30 carbon atoms, n is a number greater than 2, or a mixture thereof.

Preferably R¹ and R² each independently are hydrogen, or a substituted or unsubstituted, linear or branched, alkyl group or alkenyl group having from 1 to 30 carbon atoms, preferably from 1 to 16 carbon atoms, more preferably from 1 to 8 and most preferably from 1 to 4, or a hydroxy bearing linear or branched alkyl or alkenyl group having from 1 to 30 carbon atoms, more preferably from 1 to 16, even more preferably from 1 to 4, and most preferably R¹ and R² are methyl or hydrogen.

Preferably R³ is a substituted or unsubstituted, linear or branched, alkyl group or alkenyl group having from 1 to 30 carbon atoms, preferably from 1 to 16 carbon atoms, more preferably from 1 to 8 and most preferably from 1 to 4, or a substituted or unsubstituted, saturated or unsaturated, linear or branched aryl group having up to 30 carbon atoms, preferably from 3 to 16 and more preferably from 4 to 8 carbon atoms, or a hydroxy bearing linear or branched alkyl or alkenyl group having from 1 to 30 carbon atoms, more preferably from 1 to 16 even more preferably from 1 to 8, and most preferably R³ is butyl.

Preferably n is a number of at least 3, preferably from 3 to 2300, more preferably 3 to 100, more preferably from 3 to 20 and most preferably from 3 to 10.

The poly(alkylene glycol) alkyl ethers for use herein preferably have an average molecular weight from 164 to 100,000, more preferably from 180 to 10,000 and most preferably from 200 to 1,000.

Suitable poly(alkylene glycol) alkyl ethers for use herein include poly(propylene glycol) mono butyl ether, poly(ethylene glycol-co-propylene glycol) mono butyl ether, poly(ethylene glycol) dimethyl ether, poly(ethylene glycol-co-propylene glycol) dimethyl ether, poly(ethylene glycol) stearate or mixtures thereof. Poly(propylene glycol) mono butyl ether (average molecular weight 340) is commercially available from Aldrich or from Union Carbide under Ucon-lb 65®.

Other useful solvents for compositions of the present invention include those disclosed in U.S. Pat. Nos. 5,540,865; 5,435,935; and 5,362,422; which are hereby incorporated by reference.

A preferred type of non-aqueous, low-polarity solvent for use in the compositions herein comprises the non-vicinal C₄-C₈ branched or straight chain alkylene glycols. Materials of this type include hexylene glycol (4-methyl-2,4-pentanediol), 1,6-hexanediol, 1,3-butylene glycol and 1,4-butylene glycol. Hexylene glycol is the most preferred.

Another preferred type of non-aqueous, low-polarity solvent for use herein comprises the mono-, di-, tri-, or tetra- C₂-C₃ alkylene glycol mono C₂-C₆ alkyl ethers. The specific examples of such compounds include diethylene glycol monobutyl ether, tetraethylene glycol monobutyl ether, dipropolyene glycol monoethyl ether, and dipropylene glycol monobutyl ether. Diethylene glycol monobutyl ether, dipropylene glycol monobutyl ether and butoxy-propoxy-propanol (BPP) are especially preferred. Compounds of the type have been commercially marketed under the trade names Dowanol, Carbitol, and Cellosolve.

Another preferred type of non-aqueous, low-polarity organic solvent useful herein comprises the lower molecular weight polyethylene glycols (PEGs). Such materials are those having molecular weights of at least about 150. PEGs of molecular weight ranging from about 200 to 600 are most preferred.

Enzymes—The compositions of the present invention may optionally, but preferably, comprise one or more enzymes.

Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, βglucanases, arabinosidases, hyaluronidase, chondroitinase, laccase, mannanases, more preferably plant cell wall degrading enzymes and non-cell wall-degrading enzymes (WO 98/39403 A) and can, more specifically, include pectinase (WO 98/06808 A, JP10088472 A, JP10088485 A); pectolyase (WO98/06805 A1); pectin lyases free from other pectic enzymes (WO9806807 A1); chondriotinase (EP 747,469 A); xylanase (EP 709,452 A, WO 98/39404 A, WO98/39402 A) including those derived from microtetraspora flexitosa (U.S. Pat. No. 5,683,911); isopeptidase (WO 98/16604 A); keratinase (EP 747,470 A, WO 98/40473 A); lipase (GB 2,297,979 A; WO 96/16153 A; WO 96/12004 A; EP 698,659 A; WO 96/16154 A); cellulase or endoglucanase (GB 2,294,269 A; WO 96/27649 A; GB 2,303,147 A; WO98/03640 A; see also neutral or alkaline cellulases derived from chrysoporium lucknowense strain VKM F-3500D as disclosed in WO9815633 A); polygalacturonase (WO 98/06809 A); mycodextranase (WO 98/13457 A); thermitase (WO 96/28558 A); cholesterol esterase (WO 98 28394 A); or any combination thereof; and known amylases; oxidoreductases; oxidases or combination systems including same (DE19523389 A1); mutant blue copper oxidases (WO9709431 A1), peroxidases (see for example U.S. Pat. No. 5,605,832, WO97/31090 A1), mannanases (WO9711164, WO 99/09126, PCT/US00/00839); xyloglucanases (WO 98/50513, PCT/US/00/00839, WO 99/02663): laccases, see WO9838287 A1 or WO9838286 A1 or for example, those laccase variants having amino acid changes in myceliophthora or scytalidium laccase(s) as described in WO9827197 A1 or mediated laccase systems as described in DE19612193 A1), or those derived from coprinus strains (see, for example WO9810060 A1 or WO9827198 A1), phenol oxidase or polyphenol oxidase (JP10174583 A) or mediated phenol oxidase systems (WO9711217 A): enhanced phenol oxidase systems (WO 9725468 A WO9725469 A); phenol oxidases fused to an amino acid sequence having a cellulose binding domain (WO9740127 A1, WO9740229 A1) or other phenol oxidases (WO9708325 A, WO9728257 A1) or superoxide dismutases. Oxidoreductases and/or their associated antibodies can be used, for example with H₂O₂, as taught in WO 98/07816 A. Depending on the type of composition, other redox-active enzymes can be used, even, for example, catalases (see, for example JP093 16490 A). Examples of these and other such suitable enzymes and/or levels of use are disclosed in U.S. Pat. Nos. 5,705,464, 5,710,115, 5,576,282, 5,728,671, 5,707,950, and WO9828400 A2.

Particularly useful proteases are described in PCT publications: WO 95/30010; WO 95/30011; and WO 95/29979. Suitable proteases are commercially available as ESPERASE®, ALCALASE®, DURAZYM®, SAVINASE®, EVERLASE® and KANNASE® all from Novo Nordisk A/S of Denmark, and as MAXATASE®, MAXACAL®, PROPERASE® and MAXAPEM® all from Genencor International (formerly Gist-Brocades of The Netherlands).

A highly preferred protease is a carbonyl hydrolase variant having an amino acid sequence not found in nature, which is derived from a precursor carbonyl hydrolase by substituting a different amino acid for a plurality of amino acid residues at a position in said carbonyl hydrolase equivalent to position 103 of Bacillus amyloliquefaciens subtilisin in combination with a substitution of an amino acid residue with another naturally occurring amino acid residue at one or more amino acid residue positions corresponding to positions 1, 3, 4, 8, 9, 10, 12, 13, 16, 17, 18, 19, 20, 21, 22, 24, 27, 33, 37, 38, 42, 43, 48, 55, 57, 58, 61, 62, 68, 72, 75, 76, 77, 78, 79, 86, 87, 89, 97, 98, 99, 101, 102, 104, 106, 107, 109, 111, 114, 116, 117, 119, 121, 123, 126, 128, 130, 131, 133, 134, 137, 140, 141, 142, 146, 147, 158, 159, 160, 166, 167, 170, 173, 174, 177, 181, 182, 183, 184, 185, 188, 192, 194, 198, 203, 204, 205, 206, 209, 210, 211, 212, 213, 214, 215, 216, 217, 218, 222, 224, 227, 228, 230, 232, 236, 237, 238, 240, 242, 243, 244, 245, 246, 247, 248, 249, 251, 252, 253, 254, 255, 256, 257, 258, 259, 260, 261, 262, 263, 265, 268, 269, 270, 271, 272, 274 and 275 of Bacillus amyloliquefaciens subtilisin; wherein when said protease variant includes a substitution of amino acid residues at positions corresponding to positions 103 and 76, there is also a subtitution of an amino acid residue at one or more amino acid residue positions other than amino acid residue positions corresponding to positions 27, 99, 101, 104, 107, 109, 123, 128, 166, 204, 206, 210, 216, 217, 218, 222, 260, 265 or 274 of Bacillus amyloliquefaciens subtilisin; and one or more cleaning adjunct materials.

While any combination of the above listed amino acid substitutions may be employed, the preferred protease variant enzymes useful for the present invention comprise the substitution, deletion or insertion of amino acid residues in the following combinations:

(1) a protease variant including substitutions of the amino acid residues at position 103 and at one or more of the following positions 236 and 245;

(2) a protease variant including substitutions of the amino acid residues at positions 103 and 236 and at one or more of the following positions: 12, 61, 62, 68, 76, 97, 98, 101, 102, 104, 109, 130, 131. 159, 183, 185, 205, 209, 210, 211, 212, 213, 215, 217, 230, 232, 248, 252, 257, 260, 270 and 275;

(3) a protease variant including substitutions of the amino acid residues at positions 103 and 245 and at one or more of the following positions: 12, 61, 62, 68, 76, 97, 98, 101, 102, 104, 109, 130, 131, 159, 170, 183, 185, 205, 209, 210, 211, 212, 213, 215, 217, 222, 230, 232, 248, 252, 257, 260, 261, 270 and 275; and

(4) a protease variant including substitutions of the amino acid residues at positions 103, 236 and 245 and at one or more of the following positions: 12, 61, 62, 68, 76, 97, 98, 101, 102, 104, 109, 130, 131, 159, 183, 185, 205, 209, 210, 211, 212, 213, 215, 217, 230, 232, 243, 248, 252, 257, 260, 270 and 275, as described in the patent applications of C. Ghosh, et al, entitled “Cleaning Compositions Containing Multiply-Substituted Protease Variants” having U.S. Ser. No. 09/529905, filed Oct. 23, 1998.

Examples of commercial α-amylases products are Purafect Ox Am® from Genencor and Termamyl®, Ban® , Fungamyl® and Duramyl®, all available from Novo Nordisk A/S Denmark. WO95/26397 describes other suitable amylases: α-amylases characterised by having a specific activity at least 25% higher than the specific activity of Termamyl® at a temperature range of 25° C. to 55° C. and at a pH value in the range of 8 to 10, measured by the Phadebas® α-amylase activity assay. Suitable are variants of the above enzymes, described in WO96/23873 (Novo Nordisk). Other amylolytic enzymes with improved properties with respect to the activity level and the combination of thermostability and a higher activity level are described in WO95/35382.

Preferred selections are influenced by factors such as pH-activity and/or stability optima, thermostability, and stability to active detergents, builders and the like.

Other Adjunct Ingredients

Other known adjunct ingredients may be incorporated into the compositions of the present invention. Nonlimiting examples of such other adjunct ingredients include other solvents, other perfumes, builders, bleaches, hydrogen peroxide sources, preformed peracids, bleach activators, bleach catalysts, bleach boosters, enzyme stabilizing systems, chelants, optical brighteners, soil release polymers, dye transfer agents, dispersants, suds suppressors, suds boosting agents, dyes, colorants, filler salts, hydrotropes, photoactivators, fluorescers, fabric conditioners, hydrolyzable surfactants, perservatives, anti-oxidants, anti-shrinkage agents, anti-wrinkle agents, softening agents, other antimicrobial agents, germicides, fungicides, color speckles, silvercare, anti-tarnish and/or anti-corrosion agents, alkalinity sources, solubilizing agents, carriers, processing aids, pigments, dye fixing agents, crystal growth inhibiting agents and pH control agents and mixtures thereof.

Other Antimicrobial Agents—The compositions of the present invention may optionally comprise one or more other antimicrobial agents. Suitable other antimicrobial agents include, but are not limited to, alkylbenzyldimethylammonium chloride, dialkyldimethylammonium chloride, isopropanol, propylene glycol, hypochlorite, organic acids such as citric acid, bases such as hydroxide, hydrogen peroxide, triclosan and biguanides.

A nonlimiting list of suitable other antimicrobial agents for use in the compositions of the present invention follows:

3′,4,4′-trichloro-2-hydroxydiphenyl ether; 4,4′-dichloro-2-hydrocydiphenyl ether; 4-chloro-4′-iodo-2-hydroxydiphenyl ether; 4-chloro-4′-fluoro-2-hydroxydiphenyl ether; 4-chloro-4′-bromo-2-hydroxydiphenyl ether; 3,5′,4-tribromo-2-hydroxydiphenyl ether; 4-bromo-4′-chloro-2-hydroxydiphenyl ether; 4-bromo-2′,4-dichloro-2-hydroxydiphenyl ether; 4,4′-dibromo-2-hydroxydiphenyl ether; 4,2′,4′-trichloro-2-hydroxydiphenyl ether; 4,4′,5′-trichloro-2-hydroxydiphenyl ether; 2,6-dimethyl-4-hydroxychlorobenzene; 3,4,4′-trichlorocarbanilide; 3-trifluoromethyl-4,4′-dichlorocarbanilide; 2,2′-dihydroxy-3,3′,5,5′,6,6′-hexachlorodiphenylmethane; 2,2′-dihydroxy-3,3′,5,5′-tetrachlorodiphenylmethane; 2,2′-dihydroxy-3,3′-dibromo-5,5′-dichlorodiphenylmethane; 1-hydroxy-4-methyl-6-(2,4,4′-trimethylpentyl)-2-(1H)-pyridinone; Benzalkonium chloride; Benzethonium chloride; Carbolic acid; 1,6-di(4′-chlorophenyl-diguanido)hexane); Cresylic acid; 5-amino-1,3-bis(2-ethylhexyl)-5-methylhexahydropyrimidine); Iodophors; Methylbenzethonium chloride; Povidone-Iodine; Tetramethylthiuran disulfide; Tribrominated salicylanilide; 2-bromo-2-nitropropane-1,3-diol; cis Isomer of 1-(3-chloroallyl)-3,5,7-triaza-1-azoniaadamantane-chloride; 3-Iodo-2-propynyl butyl carbamate; Dimethyloldimethylhydantoin; 3-iodo-2-propynyl butyl carbamate+Hydantoin; Formaldehyde; Diazolidinyl Urea; Imidazolidinyl Urea; Glutaraldehyde; Kathon CG; Methyl paraben; Ethyl paraben; Propyl paraben; Butyl paraben; O-Benzyl-p-chlorophenol; O-phenylphenol; Sodium Phenylphenol; Fentichlor; 2-Phenoxyethanol; Salicylic acid, Halogenated salicylic acids; Sorbic acid; Amphoteric surfactants; Cationic surfactants; Amine oxide; Halogenated phenols; 2-pyridinethiol-1-oxide; 5,7-diiodo-8-hydroxyquinoline; and Salts of the afore mentioned antimicrobials

A wide range of quaternary compounds can also be used as antimicrobial actives. Non-limiting examples of useful quaternary compounds include: (1) benzalkonium chlorides and/or substituted benzalkonium chlorides such as commercially available Barquat® (available from Lonza), Maquat® (available from Mason), Variquat® (available from Witco/Sherex), and Hyamine® (available from Lonza): (2) di(C₆-C₁₄)alkyl di short chain (C₁₋₄ alkyl and/or hydroxyalkl) quaternary such as Bardac® products of Lonza, (3) N-(3-chloroallyl) hexaminium chlorides such as Dowicide® and Dowicil® available from Dow; (4) benzethonium chloride such as Hyamine® 1622 from Rohm & Haas; (5) methylbenzethonium chloride represented by Hyamine® 10X supplied by Rohm & Haas, (6) cetylpyridinium chloride such as Cepacol chloride available from of Merrell Labs. Examples of the preferred dialkyl quaternary compounds are di(C8-C₁₂)dialkyl dimethyl ammonium chloride, such as didecyldimethylammonium chloride (Bardac 22), and dioctyldimethylammonium chloride (Bardac 2050). Typical concentrations for biocidal effectiveness of these quaternary compounds range from about 0.001% to about 0.8%, preferably from about 0.005% to about 0.3%, more preferably from about 0.01% to about 0.2%, and even more preferably from about 0.03% to about 0.1%, by weight of the usage composition. The corresponding concentrations for the concentrated compositions are from about 0.003% to about about 2%, preferably from about 0.006% to about 1.2%, and more preferably from about 0.1% to about 0.8% by weight of the concentrated compositions.

Nonlimiting examples of suitable organic acids that can be used in the compositions of the present invention include citric, malic, succinic, and benzoic, which, when used in suitable concentrations, as further described herein, are highly efficacious against microbes, such as Salmonella choleraesuis and Staphylococcus aureus. When used in the presence of a surfactant, preferably a nonionic surfactant such as alcohol ethoxylates (for example, ALFONIC® 810-6 Ethoxylated available from Vista Chemical Company in Houston, Tex.), these acids were found to have effective residual antimicrobial activity against a variety of microbes, including gram negative (−) bacteria, such as Salmonella choleraesius, and gram positive (+) bacteria, such as Staphylococcus aureus. In general, the water soluble carboxylic acids useful in accordance with the invention have the following structure:

R—COOH

wherein R may be represented by: lower alkyl; substituted lower alkyl; hydroxy lower alkyl (e.g. HOCH₂—); carboxy lower alkyl (e.g. HOOC—CH₂—CH₂—); carboxy, hydroxy lower alkyl (e.g., HOOCCH₂ CHOH—); carboxy, halo lower alkyl (e.g. HOOCCH₂CHBr—); carboxy, dihydroxy lower alkyl (e.g. HOOC—CHOH—CHOH—); dicarboxy, hydroxy lower alkyl (e.g. HOOC—CH₂C—C(OH)(COOH) H₂—); lower alkenyl, carboxy lower alkenyl (e.g. HOOCCH═CH—); dicarboxy lower alkenyl (e.g. HOOC—CH₂C(COOH)═CH—); phenyl (C₆ H₅—); substituted phenyl (e.g. hydroxy phenyl HO—C₆ H₄—). Other acid examples include hydroxy lower alkyl e.g. lactic; carboxy. hydroxy lower alkyl, e.g. 2-methyl malic; carboxy, halo lower alkyl, e.g. 2-chloro-3-methyl succinic; carboxy, dihydroxy lower alkyl. e.g. 2-methyl tartaric; dicarboxy, hydroxy lower alkyl, e.g. 2-methyl citric acid; and carboxy lower alkenyl, e.g. fumaric. The above definitions are used in an illustrative but not a limiting sense. The term “lower” as used herein refers to an acid wherein “R” contains one to six carbon atoms. The term “substituted” indicates that one or more hydrogen atoms are substituted by halogen atoms (F, Cl, Br, I) hydroxyl groups, amino groups, thiol groups, nitro groups, cyano groups, and the like. Examples of preferred antimicrobial organic acids include, but are not limited to, citric acid, lactic acid, malic acid, salicylic acid, acetic acid, and mixtures thereof.

In a preferred embodiment, the compositions comprise organic acid at a level of from about 0.5% to about 20%, more preferably from about 1% to about 10%, and still more preferably from about 1.5% to about 7.5% by weight of the antimicrobial composition. Citric acid is a highly preferred organic acid having antimicrobial action. Citric acid is preferred because it is a natural acid and is relatively safe for use on household surfaces, especially surfaces used for food preparation such as countertops in kitchens and dining rooms. In addition, when citric acid is allowed to remain on a surface, it tends to provide a glossy or shiny film on the surface which can be aesthetically satisfying to consumers and provide a visual signal to consumers that the surface has residual antimicrobial protection. Also, the glossy film allows the consumer to identify areas on the hard surface which were inadvertently missed in treating the surface and allows the consumer to verify that an entire area has been treated.

Bacteria

The substrates/articles used in accordance with the present invention contain bacteria. The bacteria may be any bacteria known to those skilled in the art. The bacteria may be gram negative or gram positive. Preferably, the bacteria is selected from the group consisting of: Staphylococcus aureus, Staphylococcus haemolyticus, Staphylococcus capitis, Klebsiella pneumoniae, Proteus mirabilis, Serratia marcescens, Staphylococcus epidermis, Salmonella typhimurium, Shigella dysenteriae, Streptococcus faecalis, Streptococcus pyogenes, Corynebacterium xerosis, Micrococcus varians, Micrococcus luteus, Peptostreptococcus anaerobius, Propionibacterium acnes, Propionibacterium avidum, Propionibacterium granulosum, Escherichia coli, Salmonella choleraesius, Listeria monocytogenes, Enterococcus hirae and mixtures thereof, more preferably, Escherichia coli, Salmonella choleraesius, Listeria monocytogenes and mixtures thereof, and most preferably Escherichia coli, Salmonella chloleraesius and mixtures thereof.

Methods of the Present Invention

A method for bacteria-reducing a bacteria-containing substrate/article, such as those substrates/articles disclosed hereinbefore, comprising contacting the substrate/article with a bacteria-reducing system and/or antibacterial composition in accordance with the present invention, such that bacteria on or in a bacteria-containing substrate is reduced (i.e., rendered inactive, killed, etc.).

Bacteria-Reduced Substrate/Article

A bacteria-reduced substrate/article results the methods of the present invention.

Product/Instructions of Use

This invention also may encompass the inclusion of instructions on the use of the bacteria-reducing systems and/or antibacterial compositions described herein with the packages containing the bacteria-reducing systems and/or antibacterial compositions or with other forms of advertising associated with the sale or use of the bacteria-reducing systems and/or antibacterial compositions. The instructions may be included in any manner typically used by consumer product manufacturing or supply companies. Examples include providing instructions on a label attached to the container holding the system and/or composition; on a sheet either attached to the container or accompanying it when purchased; or in advertisements, demonstrations, and/or other written or oral instructions which may be connected to the purchase or use of the bacteria-reducing systems and/or antibacterial compositions.

Specifically the instructions will include a description of the use of the bacteria-reducing system and/or antibacterial composition. The instructions, for instance, may additionally include information relating to the recommended amount of antibacterial composition and/or bacteria-reducing system to apply to the bacteria-containing substrate/article, the recommended amount of the antibacterial composition and/or bacteria-reducing system to add to a solution, preferably a surfactant- and/or solvent-, including water, and/or perfume- and/or enzyme-containing solution containing a substrate/article to be treated, if soaking or rubbing is appropriate to the substrate/article; the recommended amount of water, if any, to apply to the substrate/article before and after treatment; other recommended treatment.

The bacteria-reducing system and/or antibacterial composition may be incorporated into a product, the product may be a kit comprising the bacteria-reducing system and/or antibacterial composition. Accordingly, a product comprising a bacteria-reducing system and/or antibacterial composition of the present invention, the product further including instructions for using the bacteria-reducing system and/or antibacterial composition to reduce and/or kill bacteria on and/or in a bacteria-containing substrate/article.

Nonlimiting examples of suitable products and/or compositions and/or bacteria-reducing systems in which the antibacterial agents may be used may be in any product form known to those of ordinary skill in the art, such as granules, powder, paste, foam, tablets, dimple tablets, bars, sprays, liquids, dryer-added forms, impregnated sheets, coated sheets, gels, etc. The products and/or compositions and/or bacteria-reducing systems in which the antibacterial agents may be used include, but are not limited to, heavy duty liquid compositions (TIDE commercially available from The Procter & Gamble Company), light duty liquid compositions (i.e. DAWN commercially available from The Procter & Gamble Company), heavy duty granule or powder compositions (i.e., TIDE commercially available from The Procter & Gamble Company), automatic dishwashing compositions (i.e. CASCADE commercially available from The Procter & Gamble Company). household cleaning compositions (i.e., MR. CLEAN commercially available from The Procter & Gamble Company). household deodorizing compositions (i.e., FEBREZE commercially available from The Procter & Gamble Company), produce washing compositions (i.e., FIT commercially available from The Procter & Gamble Company), fabric treatment compositions (i.e., DRYEL commercially available from The Procter & Gamble Company), cleaning/sanitizing wipes (i.e. MR CLEAN WIPES commercially available from The Procter & Gamble Company), fabric care compositions (i.e. DOWNY and/or VIBRANT commercially available from The Procter & Gamble Company)

The following examples are illustrative of the present invention, but are not meant to limit or otherwise define its scope. All parts, percentages and ratios used herein are expressed as percent weight unless otherwise specified.

### FORMULATION EXAMPLES

Dishwashing Compositions

A. Liquid dishwashing detergents of the present invention are as follows:

| | Example | Example | Example | Example | Example | Example |
|---|---|---|---|---|---|---|
| | A | B | C | D | E | F |
| | (Neat) | (Usage) | (Neat) | (Neat) | (Usage) | (Usage) |
| AE0.6S | 26.1 | 0.261 | 26.1 | 13.05 | 0.261 | 0.1305 |
| Amine oxide | 6.5 | 0.065 | 0 | 0 | 0 | 0 |
| Nonionic | 3 | 0.03 | 3 | 1.5 | 0.03 | 0.015 |
| surfactant | | | | | | |
| Suds boosting | 0.2 | 0.002 | 0.2 | 0 | 0.002 | 0 |
| polymer | | | | | | |
| Diamine | 0.5 | 0.005 | 0.5 | 0 | 0.005 | 0 |
| Sodium cumene | 3.5 | 0.035 | 3.5 | 1.75 | 0.020 | 0.0175 |
| sulphonate | | | | | | |
| sodium chloride | — | 0.005 | 0.5 | 0.25 | 0.006 | 0.0025 |
| propylene glycol | 9.8 | — | 10.0 | 5.0 | — | 0.050 |
| polypropylene | — | 0.010 | 1.0 | 0.5 | 0.010 | 0.005 |
| glycol | | | | | | |
| Citrate | 2.6 | — | — | — | — | — |
| Mg²⁺ | — | — | — | — | 0.0004 | — |
| Protease | — | — | 0.015 | 0.0075 | — | 0.000075 |
| Antibacterial | 10 | 5 | 2.5 | 0.001 | 0.01 | 0.05 |
| agent | | | | | | |
| Ethanol | — | 0.070 | 0.0 | 0.0 | 0.070 | 0.0 |
| Mole ratio | 23:8:1 | 23:8:1 | 23:8:1 | 23:8:1 | 23:8:1 | 23:8:1 |
| anionic:amine | | | | | | |
| oxide:diamine | | | | | | |
| pH @ 10% | 9 | 9 | 9 | 9 | 9 | 9 |

B. Automatic dishwashing compositions in accordance with the present invention are prepared as follows:

| Component | A | B | C |
|---|---|---|---|
| Citric Acid | 15.0 | — | — |
| Citrate | 4.0 | 29.0 | 15.0 |
| Acrylate/methacrylate copolymer | 6.0 | — | 6.0 |
| Acrylic acid maleic acid copolymer | — | 3.7 | — |
| Dry add carbonate | 9.0 | — | 20.0 |
| Alkali metal silicate | 8.5 | 17.0 | 9.0 |
| Paraffin | — | 0.5 | — |
| Benzotriazole | — | 0.3 | — |
| Termamyl 60T | 1.6 | 1.6 | 1.6 |
| Antibacterial Agent | 0.9 | 2.3 | 8 |
| Percarbonate (AvO) | 1.5 | — | — |
| Perborate monohydrate | — | 0.3 | 1.5 |
| Perborate tetrahydrate | — | 0.9 | — |
| Tetraacetylethylene diamine | 3.8 | 4.4 | — |
| Diethylene triamine penta methyl phosphonic acid | 0.13 | 0.13 | 0.13 |
| (Mg salt) | | | |
| Alkyl ethoxy sulphate - 3 times ethoxylated | 3.0 | — | — |
| Alkyl ethoxy propoxy nonionic surfactant | — | 1.5 | — |
| Suds suppressor | 2.0 | — | — |
| Olin SLF18 nonionic surfactant | — | — | 2.0 |

| | |
|---|---|
| Sulphate | Balance to 100% |

C. Dimple Tablet Automatic Dishwashing Compositions in accordance with the present invention are as follows:

| Component | A (% R.M.) | B (g R.M.) | C (g R.M.) |
|---|---|---|---|
| Tablet Body | | | |
| Sodium Carbonate | 15.348 | 3.500 | 5.25 |
| STPP (12% H₂O) | 46.482 | 10.600 | 9.93 |
| Gran HEDP | 0.789 | 0.180 | 0.28 |
| SKS 6 | 6.578 | 1.500 | 2.25 |
| 2 ratio Silicate | 7.016 | 1.600 | 1.65 |
| PB1 | 10.743 | 2.450 | 3.68 |
| Termamyl 2x PCA | 0.491 | 0.112 | .17 |
| Savinase | 0.526 | 0.120 | 0.18 |
| Plurafac | 3.508 | 0.800 | 0.9 |
| BTA | 0.263 | 0.060 | 0.09 |
| PEG | 1.140 | 0.260 | — |
| PEG 4000 | — | — | 0.39 |
| Winog | 0.439 | 0.100 | 0.15 |
| Antibacterial Agent | 0.5 | 1.3 | — |
| Perfume | 0.101 | 0.023 | 0.01 |
| Dimple Filling | | | |
| Citric Acid | 0.987 | 0.225 | 0.23 |
| Bicarbonate | 2.600 | 0.593 | 0.59 |
| Sandolan EHRL Dye | 0.007 | 0.0017 | 0.0017 |
| PEG 400/4000 | 0.395 | 0.090 | |
| PEG 400 | — | — | 0.02 |
| PEG 4000 | — | — | 0.08 |
| Antibacterial Agent | — | — | 2.0 |
| Amylase | 1.412 | 0.322 | 0.32 |
| Protease | 0.05 | 0.268 | 0.27 |

Laundry Compositions

A. Heavy duty liquid detergents of the present invention are as follows:

| | Example | Example | Example | Example | Example | Example | Example |
|---|---|---|---|---|---|---|---|
| | A | B | C | D | E | F | G |
| | (Neat) | (Neat) | (Neat) | (Neat) | (Neat) | (Neat) | (Neat) |
| AE0.6S | 5.1 | — | 1.2 | 1.3 | 0.32 | — | 6.6 |
| LAS | 7.8 | 11.0 | 23.6 | 10.6 | 13.71 | 2.4 | 9.0 |
| Nonionic | 4.8 | 4.1 | 12.0 | 5.2 | 5.16 | 12.1 | 1.2 |
| Builder | 1.0 | 1.1 | 2.1 | 1.9 | 1.34 | 0.05 | — |
| Hydrotrope | — | 1.1 | — | 0.8 | 1.23 | 0.38 | — |
| Antibacterial | 0.25 | 3 | 0.8 | 0.1 | 10 | 5 | 0.4 |
| Agent | | | | | | | |
| Brightener | 0.33 | 0.19 | 0.15 | 0.20 | 0.33 | 0.16 | 0.13 |
| Enzyme | — | — | 0.0288 | — | — | — | — |
| | AU/g | | | | | | |
| Solvent | — | — | 11.4 | 2.1 | — | 2.0 | 2.7 |
| Water | Balance | Balance | Balance | Balance | Balance | Balance | Balance |
| pH @ 25% | 9.96 | 11.36 | 9.28 | 8.39 | 11.50 | 10.05 | 7.38 |

B. Heavy duty liquid detergents of the present invention are as follows:

| Component | A | B | C | D | E |
|---|---|---|---|---|---|
| Prolease | 0.05 | 0.03 | 0.30 | 0.03 | 0.10 |
| Antibacterial Agent | 1 | 5 | 0.3 | 0.1 | 0.2 |
| C₁₂-C₁₄ alkyl sulfate, Na | 20.00 | 20.00 | 20.00 | 20.00 | 20.00 |
| 2-Butyl octanoic acid | 5.00 | 5.00 | 5.00 | 5.00 | 5.00 |
| Sodium citrate | 1.00 | 1.00 | 1.00 | 1.00 | 1.00 |
| C₁₀ alcohol ethoxylate (3) | 13.00 | 13.00 | 13.00 | 13.00 | 13.00 |
| Monethanolamine | 2.50 | 2.50 | 2.50 | 2.50 | 2.50 |

| | |
|---|---|
| Water/propylene glycol/ethanol | balance to 100% |
| (100:1:1) | |

C. Heavy duty liquid detergents of the present invention are as follows:

| | Formula 1 | Formula 2 |
|---|---|---|
| | (Wt %) | (Wt %) |
| C₁₃₋₁₅ EO7 ethoxylated surfactant | 20 | |
| C₁₂₋₁₄ amineoxide surfactant | 5 | |
| HLAS | | 20 |
| Citric acid | 6 | |
| C₁₂₋₁₈ fatty acid | | 15 |
| Diethylene triamine pentamethylene | 0.4 | |
| phosphonic acid | | |
| Hydroxyethanedimethylenephosphonic acid | 0.45 | |
| Ethoxylated polyethylene imine | 2.65 | |
| Boric acid | 2 | |
| CaCl₂ | 0.02 | 0.02 |
| Propanediol | 18 | 20 |
| Ethanol | 1 | |
| Monoethanolamine | to pH 8.5 | |
| NaOH | | to pH 8.5 |
| Protease enzyme | 0.77 | |
| Amylase enzyme | 0.06 | 0.06 |
| Cellulase enzymes | 0.16 | 0.16 |
| Cationic Silicone Polymer | 0 | 2.5 |
| Antimicrobial Agent | 1.0 | 0.5 |
| Water | to 100 parts | to 100 parts |

D. Dual compartment heavy duty liquid detergents of the present invention are as follows:

| | |
|---|---|
| First Compartment | |
| MEA | 1.10 |
| C10 APA | 0.50 |
| Na C25 AE1.80S | 19.35 |
| Propylene Glycol | 7.50 |
| Neodol 23-9 | 0.63 |
| FWA-15 | 0.15 |
| Na Toluene Sulfonate | 2.25 |
| NaOH | 2.79 |
| N-Cocoyl N-Methyl Glucamine | 2.50 |
| Citric Acid | 3.00 |
| C12-16 Real Soap | 2.00 |
| Borax Premix | 2.50 |
| EtOH | 3.25 |
| Ca Formate | 0.09 |
| Polyethyleneimine (MW 600) | 1.30 |
| ethoxylated and average of 20 times | |
| per nitrogen | |
| Ethoxylated Tetraethylene-Pentaimine | 0.60 |
| Na Formate | 0.115 |
| Fumed Silica Premix | 0.0015 |
| Soil Release Polymer | 0.08 |
| Water | 46.08 |
| Blue Liquitint 65 | 0.016 |
| Protease | 1.24 |
| Cellulase | 0.043 |
| Amylase | 0.15 |
| Silicone | 0.119 |
| Neptune LC | 0.35 |
| DTPA | 0.30 |
| Sodium Bicarbonate | 2.00 |
| Antimicrobial Agent | 1.5 |
| Second Compartment | |
| NaOH | 3.46 |
| Citric Acid | 5 |
| Preformed Peracid | 22.0 |
| Xanthan Gum | 0.45 |
| Water | Balance |

E. Heavy Duty Granular/Powder detergent compositions in accordance with the present invention are as follows:

| Component | A | B | C | D |
|---|---|---|---|---|
| Protease | 0.10 | 0.20 | 0.03 | 0.05 |
| Antibacterial Agent | 3 | 7 | 0.2 | 0.1 |
| C₁₂ alkyl benzene sulfonate | 12.00 | 12.00 | 12.00 | 12.00 |
| Zeolite A (1-10 micrometer) | 26.00 | 26.00 | 26.00 | 26.00 |
| C₁₂-C₁₄ secondary (2.3) alkyl sulfate. | 5.00 | 5.00 | 5.00 | 5.00 |
| Na salt | | | | |
| Sodium citrate | 5.00 | 5.00 | 5.00 | 5.00 |
| Optical brightener | 0.10 | 0.10 | 0.10 | 0.10 |
| Sodium sulfate | 17.00 | 17.00 | 17.00 | 17.00 |

| | |
|---|---|
| Fillers, water, minors | balance to 100% |

F. Heavy Duty Granular/Powder detergent compositions in accordance with the present invention are as follows:

| Component | A | B | C |
|---|---|---|---|
| Base Granule Components | | | |
| LAS/AS/AES (65/35) | 9.95 | — | — |
| LAS/AS/AES (70/30) | — | 12.05 | 7.70 |
| Alumino silicate | 14.06 | 15.74 | 17.10 |
| Sodium carbonate | 11.86 | 12.74 | 13.07 |
| Sodium silicate | 0.58 | 0.58 | 0.58 |
| NaPAA Solids | 2.26 | 2.26 | 1.47 |
| PEG Solids | 1.01 | 1.12 | 0.66 |
| Brighteners | 0.17 | 0.17 | 0.11 |
| DTPA | — | — | 0.70 |
| Sulfate | 5.46 | 6.64 | 4.25 |
| DC-1400 Deaerant | 0.02 | 0.02 | 0.02 |
| Moisture | 3.73 | 3.98 | 4.33 |
| Minors | 0.31 | 0.49 | 0.31 |
| B.O.T. Spray-on | | | |
| Nonionic surfactant | 0.50 | 0.50 | 0.50 |
| Agglomerate Components | | | |
| LAS/AS (25/75) | 11.70 | 9.60 | 10.47 |
| Alumino silicate | 13.73 | 11.26 | 12.28 |
| Carbonate | 8.11 | 6.66 | 7.26 |
| PEG 4000 | 0.59 | 0.48 | 0.52 |
| Moisture/Minors | 4.88 | 4.00 | 4.36 |
| Functional Additives | | | |
| Sodium carbonate | 7.37 | 6.98 | 7.45 |
| Perborate | 1.03 | 1.03 | 2.56 |
| AC Base Coating | — | 1.00 | — |
| NOBS | — | — | 2.40 |
| Soil release polymer | 0.41 | 0.41 | 0.31 |
| Cellulase | 0.33 | 0.33 | 0.24 |
| Protease | 0.1 | 0.05 | 0.15 |
| Antibacterial Agent | 0.1 | 10 | 3 |
| AE-Flake | 0.40 | 0.40 | 0.29 |
| Liquid Spray-on | | | |
| Perfume | 0.42 | 0.42 | 0.42 |
| Noionic spray-on | 1.00 | 1.00 | 0.50 |

| | |
|---|---|
| Minors | Up to 100 |

In-Dryer Compositions

A. Fabric cleaning/refreshment compositions especially suitable for use in a dryer according to the present invention, preferably for use in a containment bag, are prepared as follows:

| Ingredient | I %(wt.) | II %(wt.) | III %(wt.) | IV %(wt) |
|---|---|---|---|---|
| Water | 97.63 | 98.85 | 77.22 | 96.71 |
| Perfume | 0 | 0.38 | 0.38 | 0 |
| Surfactant | 0.285 | 0 | 0 | 0.285 |
| Solvent (e.g. BPP) | 2.0 | 0 | 0 | 2.0 |
| KATHON ® | 0.0003 | 0 | 0 | 0 |
| Emulsifier (TWEEN 20)* | 0 | 0.5 | 0.38 | 0 |
| Amine Oxide | 0.0350 | 0 | 0 | 0.0350 |
| MgCl₂ | 0.045 | 0 | 0 | 0 |
| MgSO₄ | 0 | 0 | 0.058 | 0 |
| Hydrogen Peroxide | 0 | 0 | 0 | 0.6 |
| Citric Acid | 0 | 0 | 0 | 0.05 |
| Proxel GXL | 0 | 0.08 | 0.08 | 0 |
| Bardac 2250 | 0 | 0.2 | 0.2 | 0 |
| Antibacterial Agent | 5 | 0.5 | 0.1 | 9 |
| 1,2-Propanediol | 0 | 0 | 21.75 | 0 |

*Polyoxyethylene (20) sorbitan monolaurate available from ICI Surfactants.

B. A fabric softener composition in sheet form in accordance with the present invention is as follows:

| Components | Wt. % |
|---|---|
| Co-softener* | 20.34 |
| Glycosperse S-20 | 14.67 |
| DEEHMAMS | 34.12 |
| Tallow fatty acid | 8.53 |
| (C.sub.16-18, IV = 42) | |
| added partway through DEEHMAMS | |
| quaternization | |
| Perfume/Cyclodextrin | 17.21 |
| Complex | |
| Clay** | 3.01 |
| Free Perfume | 1.45 |
| LAS | 0.67 |

Glycosperse S20 is polyethoxylated sorbitan monostearate, from Lonza, which contains about 20 ethoxylate moieties per molecule.
DEEHMAMS is di(C.sub.16-18 unsaturated ethylester)hydroxyethylmethylammonium methylsulfate.
*1:2 ratio of stearyldimethylamine:triplepressed stearic acid.
**Calcium bentonite clay, Bentolite L. sold by Southern Clay Products, or Gelwhite GP clay.

Hard Surface Cleaning Compositions

A. Formulations in accordance with the present invention that are especially suitable as hard surface cleaning compositions are as follows:

| | |
|---|---|
| I | II |

| | |
|---|---|
| Ingredients | Wt % |

| | | |
|---|---|---|
| Sodium C₁₂₋₁₄alkyl sulfate | 0.20% | — |
| Alkylpolyglucoside | — | 0.25% |
| poly(4-vinylpyridine N- | 0.075% | 0.075% |
| oxide) polymer | | |
| Sodium carbonate | 0.015% | — |
| Antibacterial Agent | 0.5 | 1 |
| Water | Balance | Balance |
| Perfume | — | — |

Fruit and Vegetable Cleaning Compositions

A. A concentrated acidic cleaning composition is prepared by dissolving the following ingredients in water.

| Ingredient | % (wt.) |
|---|---|
| PLURAFAC RA-20 | 4.5 |
| Oleic acid | 0.25 |
| Citric acid | 2.0 |
| Potassium citrate | 2.0 |
| Potassium sorbate | 0.1 |
| Sodium benzoate | 0.1 |
| Antibacterial Agent | 0.5 |
| Water | Balance |

Product pH 4

Fabric deodorizing compositions

A. Fabric deodorizing compositions of the present invention are as follows:

| | Example I | Example 2 |
|---|---|---|
| Ingredients | Wt. % | Wt. % |
| Methylated beta-cyclodextrin | 1.0 | 0.5 |
| alpha-Cyclodextrin | — | 0.5 |
| Ethylene glycol | 0.1 | 0.1 |
| Perfume A | 0.01 | 0.01 |
| Kathon CG | 0.001 | 0.0008 |
| Antibacterial Agent | 0.5 | 2.0 |
| Distilled Water | Balance | Balance |

Liquid Fabric Softeners

A. Liquid fabric softeners of the present invention are as follows:

| | | | | | | |
|---|---|---|---|---|---|---|
| DTOEDMAC (1) | 14% | 2% | 10% | 16% | 20% | 8% |
| DTDMAC (2) | — | 4% | 10% | — | — | — |
| Amine (3) | — | 2% | — | 6% | — | 2% |
| PDMS (4) | — | 1% | — | 0.5% | 0.5% | — |
| GMS (5) | — | 0.5% | 1% | — | 0.5% | — |
| Soil release polymer | — | — | 0.5% | — | 0.5% | 0.5% |
| Perfume | 0.8% | 0.5% | 0.8% | 0.7% | 0.8% | 0.3% |
| Antimicrobial Agent | 0.2% | 1.2% | 3.0% | 0.5% | 1.0% | 2.0% |
| HCl to pH | 3.8 | 3.8 | 3.6 | 3.8 | 3.6 | 3.8 |

| | |
|---|---|
| Minors & water | balance |

(1) N;N-di(2tallowyloxy-2-oxo-ethyl)-N,N-dimethylammonium chloride
(2) ditallowdimethylammonium chloride
(3) Itallowamidoethyl-2-tallowimidazoline or monotallowdipolyethoxyamine
(4) polydimethylsiloxane, having a viscosity of 800 centistokes
(5) glyceryl monostearate

While particular embodiments of the subject invention have been described, it will be obvious to those skilled in the art that various changes and modifications of the subject invention can be made without departing from the spirit and scope of the invention. It is intended to cover, in the appended claims, all such modifications that are within the scope of the invention.

The compositions of the present invention can be suitably prepared by any process chosen by the formulator, non-limiting examples of which are described in U.S. Pat. No. 5,691,297 Nassano et al., issued Nov. 11, 1997; U.S. Pat. No. 5,574,005 Welch et al., issued Nov. 12, 1996; U.S. Pat. No. 5,569,645 Dinniwell et al., issued Oct. 29, 1996; U.S. Pat. No. 5,565,422 Del Greco et al., issued Oct. 15, 1996; U.S. Pat. No. 5,516,448 Capeci et al., issued May 14, 1996; U.S. Pat. No. 5,489,392 Capeci et al., issued Feb. 6, 1996; U.S. Pat. No. 5,486,303 Capeci et al., issued Jan. 23, 1996 all of which are incorporated herein by reference.

In addition to the above examples, the cleaning compositions of the present invention can be formulated into any suitable laundry detergent composition, non-limiting examples of which are described in U.S. Pat. No. 5,679,630 Baeck et al., issued Oct. 21, 1997; U.S. Pat. No. 5,565,145 Watson et al., issued Oct. 15, 1996; U.S. Pat. No. 5,478,489 Fredj et al., issued Dec. 26, 1995; U.S. Pat. No. 5,470,507 Fredj et al., issued Nov. 28, 1995; U.S. Pat. No. 5,466,802 Panandiker et al., issued Nov. 14, 1995; U.S. Pat. No. 5,460,752 Fredj et al.. issued Oct. 24, 1995; U.S. Pat. No. 5,458,810 Fredj et al., issued Oct. 17, 1995; U.S. Pat. No. 5,458,809 Fredj et al., issued Oct. 17, 1995; U.S. Pat. No. 5,288,431 Huber et al.. issued Feb. 22, 1994 all of which are incorporated herein by reference.

Having described the invention in detail with reference to preferred embodiments and the examples, it will be clear to those skilled in the art that various changes and modifications may be made without departing from the scope of the invention and the invention is not to be considered limited to what is described in the specification.

What is claimed is:

1. An antibacterial composition comprising:
A) a compound of formula I, [chemical structure]
wherein m is an integer from 0 to 4; n is an integer from 0 to 5; the sum of m+n is greater than zero; a is 0 or 1; b is 0 or 1; g is 0 or 1; when b is 0, one of a and g must be 0; Z and Z′ are independently selected from O and S; X and X′, when present, are selected from O, S, and NR¹, where R¹ is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T, when present, is selected from C═O, C═S, S═O, and SO₂; when T is S═O or SO₂, X and X′ may not be S; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and neither Z nor Z′ is S, R for that radical may be further selected from the group consisting of F, Cl, Br, I, CN, R₂N→O, NO₂; when Z or Z′ is S, R for that radical may be further selected from the group consisting of CN, R₂N→O, NO₂; when all a, b and g are 0, at least one R must be non-H; further provided that the total number of halogen atoms in the molecule excluding any present in G does not exceed two; G is H, a suitable charge balancing counterion (Mn⁺)_{1/n}, or a cleaveable group selected from the group consisting of Si((O)ₚR²)₃, where p is independently 0 or 1; C(O)_{q}((O)ₚR²)ᵣ, wherein p is independently 0 or 1 and when q is 1, r is 1, and when q is 0, r is 3; R² is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; and
B) at least one additional component selected from the group consisting of:
i) at least 1 wt % of a surfactant, wherein the ratio of the weight of the surfactant divided by the weight of said compound I is greater than or equal to 1.0;
ii) from 0.5% to 90% of a solvent whose Hildebrand solubility parameter d_{S} (cal/cm³)^{1/2} meets the following criterion: 5<d_{S}<20, wherein a 10 wt % aqueous solution of this composition has a pH≧(pKa−1) where pKa is the calculated pKa of the phenol or thiophenol of formula I, or when G is not H, the pKa of the phenol or thiophenol of formula I that results from replacing G with H;
iii) a perfume wherein the perfume has a C log P greater than or equal to 2.0.
iv) an enzyme from 0.001 to 1.0% by weight of the composition;
v) mixtures thereof.

2. The antibacterial composition according to claim 1 wherein the composition comprises at least 1 wt % of a cationic surfactant, wherein the ratio of the weight of the surfactant divided by the weight of said compound I is greater than or equal to 1.0; and wherein a 10 wt % aqueous solution of this composition has a pH less than or equal to 7.0.

3. The antibacterial composition according to claim 1 wherein the composition comprises at least two of said additional components.

4. The antibacterial composition according to claim 1 wherein the antibacterial composition further comprises one or more of the following adjunct ingredients selected from the group consisting of: other solvents, other perfumes, builders, bleaches, bleach activators, bleach catalysts, enzyme stabilizing systems, chelants, optical brighteners, soil release polymers, dye transfer agents, dispersants, suds suppressors, suds boosting agents, dyes, colorants, filler salts, hydrotropes, photoactivators, fluorescers, fabric conditioners, hydrolyzable surfactants, perservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments and pH control agents and mixtures thereof.

5. The antibacterial composition according to claim 1 wherein the enzyme is selected from the group consisting of: proteases, amylases, cellulases, mannanases, xyloglucanases, pectinases, lipases, laccases, peroxidases and mixtures thereof.

6. An antibacterial composition comprising:
A) a substituted phenol or thiophenol compound of formula II: 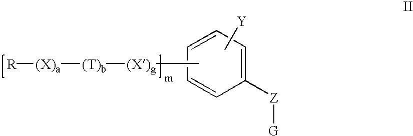
wherein m is an integer from 0 to 4; a is 0 or 1; b is 0 or 1; g is 0 or 1; when b is 0, one of a and g must be 0; Z is selected from O and S; X and X′, when present, are selected from O, S, and NR¹; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and Z is 0, R for that radical may be further selected from the group consisting of F, Cl, Br, I, CN, R₂N→), NO₂; T, when present, is selected from C═O, C═S, S═O, and SO₂; when T is S═O or SO₂, X and X′ may not be S; Y is a radical comprising at least 1 but no more than 20 carbon atoms and containing a substituent —X″—-H, where X″ is selected from O, S, and N—(T′)_{′}—(X′″)_{a′}—R², where a′ is 0 or 1, b′ is 0 or 1, and X′″, when present, is selected from O, S, and NR²; R² is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T′, when present, is selected from C═O, C═S, and SO₂; when T′ is SO₂, X′″ may not be S; G is H, a suitable charge balancing counterion (Mⁿ⁺)_{1/n}, or a cleaveable group selected from the group consisting of Si((O)ₚR³)₃, where p is independently 0 or 1; C(O)_{q}((O)ₚR³)ᵣ, wherein p is independently 0 or 1 and when q is 1, r is 1, and when q is 0, r is 3; R³ is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; the parameter d_{Z-H}, the center to center distance from the phenolic oxygen atom or the thiophenolic sulfur atom to the H atom of —X″—H, must satisfy the following criterion in at least one rotational conformation of the compound II:
1.0 Å≦d_{Z-H}≦4.0 Å;
wherein when G is H or replaced by H, the pKₐ of the substituted phenol or thiophenol, or resulting substituted phenol or thiophenol is from about 5 to about 11; and
B) a surfactant wherein the ratio of the weight of the surfactant divided by the weight of the substituted compound II is greater than or equal to 1.0 and further provided that the surfactant is 1 wt % or greater of the composition; and
C) from 0.5% to 90% of a solvent whose Hildebrand solubility parameter d_{S} (cal/cm³) meets the following criterion: 5<d_{S}<20,
further provided that a 10 wt % aqueous solution of this composition has a pH≧(pKₐ−1) where pKₐ is the calculated pKₐ of the substituted phenol or thiophenol or, when G is not H, the resulting substituted phenol or thiophenol of formula II.

7. The antibacterial composition according to claim 6 wherein the antibacterial composition exhibits Dilute Efficacy according to the Dilute Efficacy Test.

8. The antibacterial composition according to claim 6 wherein G is C(O)_{q}((O)ₚR¹)ᵣ, wherein p is independently 0 or I and when q is 1, r is 1, and when q is 0, r is 3; R¹ is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; and wherein the antibacterial composition further comprises hydrogen peroxide or hydrogen peroxide releasing agents.

9. A bacteria-reducing system comprising a compound of formula I, [chemical structure]
wherein m is an integer from 0 to 4; n is an integer from 0 to 5; the sum of m+n is greater than zero; a is 0 or 1 b is 0 or 1; g is 0 or 1; when b is 0, one of a and g must be 0; Z and Z′ are independently selected from O and S; X and X′, when present, are selected from O, S, and NR¹, where R¹ is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; T, when present, is selected from C═O, C═S, S═O, and SO₂; when T is S═O or SO₂, X and X′ may not be S; when either a, b or g is 1 for a radical R—(X)ₐ—(T)_{b}—(X′)_{g}—, R for that radical is independently selected from the group consisting of H, C₁-C₁₆ linear or branched, substituted or unsubstituted alkyl, alkenyl, alkynyl, cycloalkyl, cycloalkenyl, alkaryl, aralkyl, and aryl; when a, b and g are all 0 for a radical and neither Z nor Z′ is S, R for that radical may be further selected from the group consisting of F, Cl, Br, I, CN, R₂N→O, NO₂; when Z or Z′ is S, R for that radical may be further selected from the group consisting of CN, R2N→O, NO2; when all a, b and g are 0, at least one R must be non-H; further provided that the total number of halogen atoms in the molecule excluding any present in G does not exceed two: G is H, a suitable charge balancing counterion (Mⁿ⁺)_{1/n}, or a cleaveable group selected from the group consisting of Si((O)ₚR²)₃, where p is independently 0 or 1; C(O)_{q}((O)ₚR²)ᵣ, wherein p is independently 0 or 1 and when q is 1, r is 1, and when q is 0, r is 3; R² is independently selected from the group consisting of C₁-C₁₆ linear or branched, substituted or unsubstituted alkyl, alkenyl, alkynyl, cycloalkyl, cycloalkenyl, alkaryl, aralkyl, and aryl, and mixtures thereof; wherein the bacteria-reducing system reduces bacteria on a substrate.

10. The bacteria-reducing system according to claim 9 wherein the system further comprises at least one additional component selected from the group consisting of:
a) a surfactant;
b) a solvent:
c) a perfume having a C log P greater than or equal to 2.0;
d) an enzyme; and
e) mixtures thereof.

11. The bacteria-reducing system according to claim 10 wherein the ratio of the weight of the surfactant divided by the weight of the substituted salicylanilide compound of formula I is greater than or equal to 1.0 and further provided that the surfactant is 1 wt % or greater of the bacteria-reducing system.

12. The bacteria-reducing system according to claim 10 wherein the solvent has a Hildebrand solubility parameter d_{S} (cal/cm³)^{1/2} of: 5<d_{S}<20, and wherein a 10 wt % aqueous solution of the bacteria-reducing system has a pH≧(pKa−1) where pKa is the calculated pKa of the compound of formula I wherein G is H or replaced by H.

13. The bacteria-reducing system according to claim 9 wherein the substrate comprises a hard substrate selected from the group consisting of: utensils, dishes, cookware, pots, pans, skillets, baby bottles, baby nipples, glassware, dentures, kitchen cutting boards and mixtures thereof.

14. The bacteria-reducing system according to claim 9 wherein the substrate comprises a soft substrate selected from the group consisting of: textiles, fabrics, garments, sponges, wash cloths, brushes, gloves, scouring pads, reusable wipes, animal and/or human skin and mixtures thereof.

15. The bacteria-reducing system according to claim 9 wherein the bacteria are selected from the group consisting of: Escherichia coli, Salmonella choleraesius, Listeria monocytogenes and mixtures thereof.

16. A method for bacteria-reducing a bacteria-containing substrate comprising contacting the substrate with a bacteria-reducing system according to claim 9.

17. The method according to claim 16 wherein the substrate comprises a hard substrate selected from the group consisting of: utensils, dishes, cookware, pots, pans, skillets, baby bottles, baby nipples, glassware, dentures, kitchen cutting boards and mixtures thereof.

18. The method according to claim 16 wherein the substrate comprises a soft substrate selected from the group consisting of: textiles, fabrics, garments, sponges, wash cloths, brushes, gloves, scouring pads, reusable wipes, animal and/or human skin and mixtures thereof.

19. A bacteria-reduced substrate made by the method of claim 16.

20. A bacteria-reducing product comprising an antibacterial composition as claimed in claim 1, said product further including instructions for using said antibacterial composition to reduce bacteria on a substrate in need of treatment, the instructions including the step of contacting the substrate in need of treatment with the antibacterial composition such that said antibacterial composition treats said substrate.

21. A bacteria-reducing product comprising a bacteria-reducing system as claimed in claim 9, said product further including instructions for using said bacteria-reducing system to reduce bacteria on a substrate in need of treatment, the instructions including the step of contacting the substrate in need of treatment with the bacteria-reducing system such that said bacteria-reducing system treats said substrate.

22. The bacteria-reducing product according to claim 20 wherein said product is a liquid detergent composition.

23. The bacteria-reducing product according to claim 21 wherein said product is a liquid detergent composition.

24. An antibacterial composition according to claim 1 wherein m is an integer from 0 to 2; n is an integer from 0 to 2; g is 0; Z and Z′ are 0; and T, when present, is selected from C═O and SO₂.

25. The antibacterial composition according to claim 24 wherein the compound is selected from the group consisting of 4-chlorosalicylanilide, 5-chlorosalicylanilide and mixtures thereof.

26. The antibacterial composition according to claim 25 wherein the compound is 5-chlorosalcylanilide.
